(19) United States
(12) Patent Application Publication
(10) Pub. No.: US 2026/0256774 A1
(43) Pub. Date: Sep. 3, 2026

(54) METHODS OF TREATING OPIOID OVERDOSE TO PREVENT OR REDUCE THE RISK OF ADVERSE HEALTH EFFECTS RESULTING FROM AN OPIOID OVERDOSE

(71) Applicant: INDIVIOR UK LIMITED, Hull (GB)

(72) Inventors: Celine M. LAFFONT, North Chesterfield, VA (US); Mark ELLISON, North Chesterfield, VA (US); Phil SKOLNICK, North Chesterfield, VA (US)

(21) Appl. No.: 19/555,218

(22) Filed: Mar. 3, 2026

Related U.S. Application Data
(60) Provisional application No. 63/766,089, filed on Mar. 3, 2025.

(51) Int. Cl. A61K 31/485; A61K 9/00; A61P 25/36

(52) U.S. Cl. CPC A61K 31/485; A61K 9/0043; A61P 25/36

(57) ABSTRACT

Embodiments disclosed include methods treating opioid overdose in a human by administering to the human in need thereof an intranasal pharmaceutical formulation comprising at least one pharmaceutically acceptable carrier and consisting of a therapeutically effective amount of nalmefene opioid antagonist, to further reduce a predicted risk of incidence of an adverse health effect for the human following the administering of the intranasal pharmaceutical formulation compared to a predicted risk of incidence of the adverse health effect for the human following the administering to the human of an intranasal pharmaceutical formulation comprising a pharmaceutically acceptable carrier and consisting of a therapeutically effective amount of naloxone opioid antagonist.

### CROSS REFERENCE TO RELATED APPLICATION

This application claims priority to U.S. Provisional Patent Application No. 63/766,089, filed Mar. 3, 2025, the disclosure of which is incorporated by reference herein.

### BACKGROUND

The disclosure relates to methods of treating opioid overdose in a subject by rapid intervention using intranasal reversal agents. Specifically, the disclosure includes methods of treating opioid overdose in a subject to reduce the risk or magnitude of adverse health effects resulting from an opioid overdose including respiratory depression, cardiac arrest, brain hypoxia, and death. The disclosed methods of treatment include administration of a nasal pharmaceutical formulation including nalmefene. Embodiments include methods of treatment comprising administering a nasal pharmaceutical formulation including nalmefene, an opioid antagonist, to treat and/or reverse effects of synthetic opioid overdose compared to administration of a nasal pharmaceutical formulation of naloxone, also known as Narcan Nasal Spray.

Over the past 20 years, there has been an alarming rise in the misuse and abuse of prescription opioids in the United States. Opioids include substances such as morphine, fentanyl, codeine, hydrocodone, oxycodone, a crush resistant form of oxycodone, oxymorphone, hydromorphone, tapentadol, and methadone. Among the most prominent manifestations of what is often referred to as the ‘opioid epidemic’ are a rising number of overdose deaths (estimated at more than 33,000 in 2015) and hospital visits (estimated at more than 1.25 million) linked to opioid misuse. There has been a dramatic shift in the complexion of the opioid epidemic over the past decade due to the increased availability of illicit, high potency opioids exemplified by fentanyl. In 2023, the number of opioid overdose deaths in the United States rose to over 80,000 for the past 12 months finishing in December 2023; fatalities attributed to fentanyl and fentanyl analogs (collectively termed “synthetic opioids”) are largely responsible (>90%) for this dramatic spike and surpass both prescription opioids and heroin as the leading cause of overdose deaths. See, Ahmad F B, Cisewski J A, Rossen L M, Sutton P. Provisional drug overdose death counts. National Center for Health Statistics 2024.

Opioid overdoses, particularly those caused by high potency synthetic opioids, even if accidental, may result in fatalities. Also, periods of hypoxia associated with non-fatal overdoses have the potential to cause enduring functional impairment and brain damage making rapid restoration of breathing critical for minimizing the potential for long-term neurologic consequences. For individuals with opioid use disorder who are likely to experience recurrent episodes of prolonged opioid-induced respiratory depression, the damage is cumulative and may lead to the development of important disability, including mental and motor dysfunction. Fentanyl and its structural analogs are embedded in the illicit drug supply in the United States and the identification of other classes of synthetics (e.g., benzimidazoles such as etonitazene) in the United States and Europe represents an emerging threat. These ‘novel synthetic opioids’ (NSOs) share many of the chemical and biological properties that make fentanyl and its analogs inherently more dangerous than opium-based alkaloids such as morphine or heroin.

Multiple factors contribute to the dangers posed by fentanyl and other synthetic opioids. Synthetic opioids increase the likelihood that use will result in a fatal overdose because of two properties (a) their high potency (e.g., fentanyl has been estimated to be 40-165-fold more potent than morphine on a mg/kg basis) and (b) their high lipophilicity (synthetics are highly lipophilic molecules that penetrate the brain rapidly and have a more rapid onset of action than opium-based alkaloids such as morphine). These properties reduce the window of opportunity to successfully intervene following an overdose. Moreover, unlike naturally occurring opiates (e.g., morphine) and semi-synthetic opiates (e.g., heroin and oxycodone), synthetic opioids (hereafter referred to as “synthetics”) are piperidine derivatives with simple chemistry compared to morphinans: fentanyl contains no centers of asymmetry, and its structure is highly conducive to derivatization. Synthetics are relatively inexpensive to produce (the cost of a kg of illicit fentanyl is roughly $3,500 compared to $65,000 for heroin, Frank, Rand Pollack, H. 2017. NEJM 376:605-607) and fentanyl is −50-fold more potent than heroin Volkow, ND and Collins, F. 2017. NEJM 377:391-394. Multiple fentanyl derivatives (4-fluoroisobutyrlfentanyl, acrylfentanyl, acetylfentanyl. and 3-methylfentanyl), including the veterinary anesthetic, carfentanil (which is about 100 times more potent than fentanyl, Volkow, ND and Collins, F. 2017. NEJM 377:391-394), have been identified by the DEA in drug seizures. The potency of these illicit synthetics facilitates transport: for example, 1 kg fentanyl is equivalent to 50 kg of heroin. The lethal dose of fentanyl is considered to be approximately 2 mg, suggesting a lethal dose of carfentanil would be around 20 μg, which is no more than a few specks of compound.

Fentanyl and its derivatives are absorbed through the skin, mucous membranes, and lungs. The danger posed by these synthetics is so great that guidelines have been issued to prevent occupational exposure to emergency responders, and there are multiple reports of accidental contact with fentanyl by law enforcement officials resulting in hospitalization. Moreover, there is evidence that synthetics can be weaponized. In 2002, Russian security forces aerosolized a fentanyl derivative (likely a mixture of carfentanil and remifentanil) to immobilize Chechen terrorists who held hostages in a Moscow theater. The aerosolized gas resulted in the deaths of about 120 hostages. Multiple countries have assessed carfentanil and related synthetics for offensive and defensive applications, and there is a concern that terrorist groups can readily obtain kg quantities of these molecules.

Opioid antagonists such as naloxone and nalmefene interact at (bind to) the same receptors as opioids. Opioid antagonists bind to these receptors with high affinity, and compete with opioids (e.g., oxycodone, morphine, heroin and fentanyl) at the receptors. By binding to the receptors in place of opioids, opioid antagonists can reverse the pharmacological effects of opioids, including symptoms associated with overdose such as respiratory depression and somnolence. Opioid antagonists have been successfully used to reverse the effects of an opioid overdose.

The rapid onset of respiratory depression following a synthetic opioid overdose makes prompt intervention a necessary condition for a successful rescue. Intravenous (IV) naloxone has typically been used for the reversal of an opioid overdose because it rapidly delivers high naloxone concentrations needed to restore respiration-in the face of a potentially lethal synthetic overdose and can be titrated to effect to minimize the risk of precipitated withdrawal symptoms in patients with a history of opioid use. However, the IV option is often unavailable in a community setting where first responders rely on either intramuscular (IM) or intranasal (IN) products. Because opioid overdoses are highly heterogeneous events, studies examining the effectiveness of opioid antagonists (e.g. different doses, dosing strategies) based on case studies and chart reviews are not only subject to factors which contribute to the heterogeneous nature of an overdose (e.g. ranging from type(s) and quantity(ies) of opioid(s) taken to the latency between overdose and intervention) but also to the variability in reporting outcomes (e.g. changes in respiration versus consciousness), study quality, and selection bias.

Therefore, there is a need for an opioid antagonist to be available in a form that can be administered quickly and easily by first responders, law enforcement including the police, customs, border agents, and military personnel if exposure to opioids, especially high potency synthetics, is suspected.

### SUMMARY

The disclosure relates to a method of treating opioid overdose in a human by administering to the human in need thereof a single dose of an intranasal pharmaceutical formulation comprising at least one pharmaceutically acceptable carrier and consisting of a therapeutically effective amount of nalmefene opioid antagonist, to reduce a predicted risk of an adverse health effect resulting from the opioid overdose for the human following the administration of said intranasal pharmaceutical formulation compared to a predicted risk of said adverse health effect for the human following the administration of a single dose of an intranasal pharmaceutical formulation comprising a pharmaceutically acceptable carrier and consisting of a therapeutically effective amount of naloxone opioid antagonist.

Embodiments disclosed include an intranasal pharmaceutical formulation for treatment of opioid overdose in a human in need thereof, comprising at least one pharmaceutically acceptable carrier and consisting of a therapeutically effective amount of nalmefene opioid antagonist. The intranasal pharmaceutical formulation is configured to further reduce the predicted risk of adverse health effect from an opioid overdose compared to an intranasal pharmaceutical formulation for treatment of opioid overdose in a human in need thereof comprising at least one pharmaceutically acceptable carrier and consisting of a therapeutically effective amount of naloxone opioid antagonist.

In some embodiments disclosed, the nalmefene is nalmefene hydrochloride. In some embodiments, the nalmefene is nalmefene freebase.

In some embodiments, the adverse health effect is chosen from at least one of respiratory depression, cardiac arrest, brain hypoxia, and death.

The present disclosure may be understood more readily by reference to the following detailed description and the illustrative Example included herein. Unless defined otherwise, all technical and scientific terms used herein have the meaning commonly understood by one of ordinary skill in the relevant art.

### BRIEF DESCRIPTION OF THE FIGURES

FIG. 1 shows a flowchart that schematically represents the disclosed methods of treating opioid overdose, according to some embodiments.

FIG. 2 is a schematic representation of a protocol adopted in an example study (Study I) to evaluate the methods of treatment of opioid overdose as disclosed herein, according to an embodiment.

FIG. 3 shows graphical representations of the reversal of opioid (remifentanil)-induced respiratory depression (as measured by minute ventilation, MV, under hypercapnic conditions) following administration of an opioid antagonist (nalmefene or naloxone) using formulations and/or methods disclosed herein, in an example study (Study I), according to an embodiment.

FIG. 4A shows graphical representation of reversal of opioid-induced respiratory depression, expressed by minute ventilation, by naloxone and nalmefene, using formulations and/or methods disclosed herein, in an example study (Study I), according to an embodiment.

FIG. 4B shows graphical representation of a point estimate favoring nalmefene, demonstrating non-inferiority and superiority of IN pharmaceutical formulation including nalmefene over naloxone, in treating opioid-induced respiratory depression, using formulations and/or methods disclosed herein, in an example study (Study I), according to an embodiment.

FIG. 5 shows a graphical representation of proportion of subjects in an example study (Study I) who had extreme responses to IN pharmaceutical formulation including either nalmefene or naloxone compared to the other, administered to treat opioid overdose using formulations and/or methods disclosed herein, according to an embodiment.

FIG. 6 shows a graphical representation of effects in plasma concentration of opioid antagonists naloxone and nalmefene after administration to treat opioid overdose using formulations and/or methods disclosed herein, in an example study (Study 1), according to an embodiment.

FIG. 7 shows a schematic representation of a translational model of synthetic opioid overdose, that was expanded with intranasal nalmefene and naloxone data “Laffont Model”), to demonstrate and/or evaluate methods disclosed herein, according to some embodiments.

FIGS. 8A and 8B are graphical representations illustrating a reproducibility between simulated and clinically recorded effects of reversal of opioid-induced respiratory depression by formulations and/or methods disclosed herein, in an example study (Study II), according to example implementations.

FIGS. 9A1-9A4, 9B1-9B4, 9C1-9C4, and 9D1-9D4 show graphical representations of simulation results with the Laffont model (chronic opioid users), demonstrating treatment of adverse health effects of opioid overdose using formulations and/or methods disclosed herein, in an example study (Study II), according to an embodiment.

FIG. 10 graphically illustrates the predicted incidence of cardiac arrests following intravenous bolus doses of fentanyl in opioid-naïve subjects, without and after intervention using intranasal (IN) naloxone and IN nalmefene using methods disclosed herein, in an example study (Study III) according to some embodiments.

FIG. 11 graphically illustrates the predicted incidence of cardiac arrests following intravenous bolus doses of fentanyl in subjects who are chronic opioid users, without and after intervention using IN naloxone and IN nalmefene using methods disclosed herein, in an example study (Study III), according to some embodiments.

FIG. 12 graphically illustrates the predicted incidence of cardiac arrests following intravenous bolus doses of carfentanil in opioid-naïve subjects, without and after intervention using IN naloxone and IN nalmefene using methods disclosed herein, in an example study (Study III), according to some embodiments.

FIG. 13 graphically illustrates the predicted incidence of cardiac arrests following intravenous bolus doses of carfentanil in subjects who are chronic opioid users, without and after intervention using IN naloxone and IN nalmefene using methods disclosed herein, in an example study (Study III), according to some embodiments.

FIGS. 14A, 14B, and 14C are graphical illustrations of the percentage of simulated subjects who are opioid-naïve and experience cardiac arrests after being exposed to one of three increasing bolus doses of intravenous fentanyl, respectively. The graphs show the percentage of simulated subjects experiencing cardiac arrests as a function of delay in delivery of intervention in the form of antagonists IN naloxone and IN nalmefene using methods disclosed herein, in an example study (Study III), according to some embodiments.

FIGS. 15A, 15B, and 15C are graphical illustrations of the percentage of simulated subjects who are opioid-naïve and experience cardiac arrests after being exposed to one of three increasing bolus doses of intravenous carfentanil, respectively. The graphs show the percentage of simulated subjects experiencing cardiac arrests as a function of delay in delivery of intervention in the form of antagonists IN naloxone and IN nalmefene using methods disclosed herein, in an example study (Study III), according to some embodiments.

FIGS. 16A, 16B, and 16C are graphical illustrations of the percentage of simulated subjects who are chronic opioid users and experience cardiac arrests after being exposed to one of three increasing bolus doses of intravenous fentanyl, respectively. The graphs show the percentage of simulated subjects experiencing cardiac arrests as a function of delay in delivery of intervention in the form of antagonists IN naloxone and IN nalmefene using methods disclosed herein, in an example study (Study III), according to some embodiments.

FIGS. 17A, 17B, and 17C are graphical illustrations of the percentage of simulated subjects who are chronic opioid users and experience cardiac arrests after being exposed to one of three increasing bolus doses of intravenous carfentanil, respectively. The graphs show the percentage of simulated subjects experiencing cardiac arrests as a function of delay in delivery of intervention in the form of antagonists IN naloxone and IN nalmefene using methods disclosed herein, in an example study (Study III), according to some embodiments.

FIGS. 18A and 18B are graphical illustrations of predicted brain hypoxia and its evolution after exposure to a potentially lethal dose of 1.63 and 2.97 mg of fentanyl, respectively, in a typical (e.g., mean) simulated subject with chronic opioid use and the reversal or treatment through intervention at an identified first time in the form of antagonists IN naloxone and IN nalmefene using methods disclosed herein, in an example study (Study III), according to some embodiments.

FIGS. 19A and 19B are graphical illustrations of predicted brain hypoxia and its evolution after exposure to a potentially lethal dose of 0.012 or 0.022 mg of carfentanil, respectively, in a typical (e.g., mean) simulated subject with chronic opioid use and the reversal or treatment through intervention at an identified first time in the form of antagonists IN naloxone and IN nalmefene using methods disclosed herein, in an example study (Study III), according to some embodiments.

FIGS. 20A and 20B are graphical illustrations of predicted brain hypoxia and its evolution after exposure to a potentially lethal dose of 1.63 and 2.97 mg of fentanyl, respectively, in a typical (e.g., mean) simulated subject with chronic opioid use and the reversal or treatment through intervention at an identified second time in the form of antagonists IN naloxone and IN nalmefene using methods disclosed herein, in an example study (Study III), according to some embodiments.

FIGS. 21A and 21B are graphical illustrations of predicted brain hypoxia and its evolution after exposure to a potentially lethal dose of 0.012 or 0.022 mg of carfentanil, respectively, in a typical (e.g., mean) simulated subject with chronic opioid use and the reversal or treatment through intervention at an identified second time in the form of antagonists IN naloxone and IN nalmefene using methods disclosed herein, in an example study (Study III), according to some embodiments.

### DETAILED DESCRIPTION

Provided herein are methods for the reversal and/or treatment of opioid overdose or symptoms associated with opioid overdose in a human in need thereof comprising the administration to the human of a sterile, nasal pharmaceutical formulation containing the opioid antagonist nalmefene at one or more identified time points following detection of a suspected or confirmed opioid overdose.

Also disclosed herein are methods and an expanded mechanistic model based on a previously developed model to simulate effects of opioid overdose and effects of treating the same with IN pharmaceutical formulations comprising at least one pharmaceutically acceptable carrier and consisting of a therapeutically effective amount of an opioid antagonist that is either nalmefene or, as a comparator, naloxone; the expanded model integrates IN naloxone and IN nalmefene pharmacokinetics.

Also, disclosed are example studies examining (I) clinical data illustrating the effects of IN pharmaceutical formulations comprising nalmefene compared to naloxone; (II) development, validation, and implementation of a mechanistic model to simulate effects of opioid overdose and effects of treating the same with IN pharmaceutical formulations including opioid antagonists as disclosed herein; and (III) implementation of a mechanistic model to simulate effects of opioid overdose under varying dosing scenarios and effects of treating the same with varying delays in intervention using IN pharmaceutical formulations including opioid antagonists as disclosed herein.

There are currently FDA approved methods/devices for nasal antagonist administration to treat opioid overdose.

### Definitions

As used herein, the following terms have their meanings indicated.

Opioid receptors are G protein-coupled receptors (GPCRs) that are activated by endogenous opioid peptides, by clinically important alkaloid analgesic drugs such as morphine, and by synthetics, i.e., neither derived from nor present in opium poppies, analgesics such as methadone and fentanyl. There are three principal types of opioid receptors: the μ-opioid receptor, the δ-opioid receptor, and the κ-opioid receptor. Opioids depress respiration, which is controlled principally through medullary respiratory centers with peripheral input from chemoreceptors and other sources. Opioids produce inhibition at the chemoreceptors via μ-opioid receptors and in the medulla via μ- and possibly δ-receptors. Oxycodone and other opioid analgesics (such as hydrocodone and fentanyl) as well as heroin and methadone are all implicated in fatal overdoses.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as a variable within a range. When no range is recited, such as a margin of error or a standard deviation to a mean value given in a chart or table of data, the term “about” should be understood to mean the greater of the range which would encompass the recited value and the range which would be included by rounding up or down to that figure as well, considering significant figures, and the range which would encompass the recited value plus or minus 20%.

The term “active ingredient” or “pharmaceutically active compound” is defined in the context of a “pharmaceutical formulation” and is intended to mean a component of a pharmaceutical formulation that provides the primary pharmacological effect, as opposed to an “inactive ingredient”, which would generally be recognized as providing no pharmaceutical benefit.

The term “actuation,” as used herein, refers to operation of the device such that the pharmaceutical formulation is delivered therefrom.

The term “agonist,” as used herein, refers to a moiety that interacts with, and activates, a receptor and thereby initiates a physiological or pharmacological response characteristic of that receptor. The term “antagonist,” as used herein, refers to a moiety that interacts with or binds to a receptor at the same site as an agonist (for example, either an endogenous ligand or drug molecule) or at an allosteric site, but which does not activate the intracellular response initiated by the agonist and can thereby inhibit the intracellular responses by an agonist or partial agonist. An antagonist does not diminish the baseline intracellular response in the absence of an agonist or partial agonist.

The term “delivery time,” as used herein, refers to the amount of time that elapses between a determination made by a healthcare professional, first responder, law enforcement (e.g., police, customs, and border patrol agents), military personnel, or an untrained individual, that another individual is in need of an opioid antagonist and completion of the delivery.

The term “disease,” as used herein, is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “equivalent,” as used herein, refers to a weight of an opioid antagonist selected from nalmefene and pharmaceutically acceptable salts thereof that is equimolar to a specified weight of nalmefene hydrochloride.

The term “excipient,” as used herein, refers to a natural or synthetic substance formulated alongside the active ingredient of a medication, included for long-term stabilization, bulking up solid formulations, or to confer a therapeutic enhancement on the active ingredient in the final dosage form, such as facilitating drug absorption, reducing viscosity, or enhancing solubility.

An individual “who is at risk for accidental opioid overdose”, includes an individual who is accidentally exposed to opioids and/or anticipates incidental opioid exposure.

As used herein, two embodiments are “mutually exclusive” when one is defined to be something which is different than the other. For example, an embodiment wherein the amount of nalmefene hydrochloride is specified to be 0.5 mg is mutually exclusive with an embodiment wherein the amount of nalmefene hydrochloride is specified to be 2 mg. However, an embodiment wherein the amount of nalmefene hydrochloride is specified to be 0.5 mg is not mutually exclusive with an embodiment in which the pharmaceutical formulation comprises an aqueous solution of about 300 μL-1.0 mL nalmefene hydrochloride.

The term “naloxone,” as used herein, refers to a compound of the following structure:

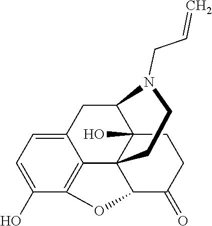

or a pharmaceutically acceptable salt, hydrate, or solvate thereof. The CAS registry number for naloxone is 465-65-6. Other names for naloxone include: 17-allyl-4,Sa-epoxy-3,14-dihydroxymorphinan-6-one; (−)-17-allyl-4,Sa-epoxy-3,14-dihydroxymorphinan-6-one; 4,Sa-epoxy-3,14-dihydroxy-17-(2-propenyl)morphinan-6-one; and (−)-12-allyl-7,7a,8,9-tetrahydro-3,7a-dihydroxy-4aH-8,9c-iminoethanophenanthro[4,5-bcd]furan-5(6H)-one. Naloxone hydrochloride may be anhydrous (CAS Reg. No. 357-08-4) and also forms a dihydrate (CAS No. 51481-60-8). It has been sold under various brand names including Narcan®, Nalone®, Nalossone®, Naloxona®, Naloxonum®, Narcanti®, and Narcon®.

The term “nalmefene,” as used herein, refers to 1 7-cyclopropylmethyl-4,5a-epoxy-6-methylenemorphinan-3,14-diol, a compound of the following structure:

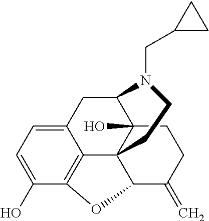

Nalmefene hydrochloride (CAS Reg. No. 58895-64-0) has been marketed under the trade names Nalmetrene®, Cervene®, Revex®, Arthrene®, and Incystene®.

Nalmefene or naloxone may optionally exist as pharmaceutically acceptable salts including pharmaceutically acceptable acid addition salts prepared from pharmaceutically acceptable non-toxic acids including inorganic and organic acids.

Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. The acid addition salts may be obtained as the direct products of compound synthesis. In the alternative, the free base may be dissolved in a suitable solvent containing the appropriate acid and the salt isolated by evaporating the solvent or otherwise separating the salt and solvent. Nalmefene and its salts may form solvates with standard low molecular weight solvents using methods known to the skilled artisan.

The term “opioid overdose,” as used herein, refers to an acute medical condition caused by voluntary or accidental exposure to an opioid agonist in a subject. Symptoms of opioid overdose include respiratory depression, central nervous system depression (which may include sedation, hypoxia, altered level consciousness, miotic (constricted) pupils), and cardiovascular depression (which may include hypoxemia and hypotension).

Visible signs of opioid overdose or suspected opioid overdose include unresponsiveness and/or loss of consciousness (unresponsive to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting.

The term “pharmaceutical formulation, or equivalently, “pharmaceutical composition,” as used herein, refers to a formulation comprising at least one active ingredient; including but not limited to nalmefene and/or salts, solvates and/or hydrates thereof, or a pharmaceutically acceptable formulation with an active ingredient consisting of nalmefene, together with at least one pharmaceutically acceptable carrier or excipient, whereby the formulation is amenable to use for a specified, efficacious outcome in a subject (for example, without limitation, a human).

The term “pharmaceutically acceptable,” as used herein, refers to a component of a pharmaceutical formulation that is compatible with the other ingredients of the formulation and not overly deleterious to the recipient thereof.

The term “receptor binding or occupancy” refers to a characterization of the binding kinetics between a drug and a receptor, and characterization of the drug binding affinity at this receptor.

The term “providing” in the context of providing a co-packaged drug product as disclosed herein to an individual includes co-packaging the drug product, prescribing the co-packaged drug product, and dispensing the co-packaged drug product. The providing may be done either directly to an individual (for example, to an individual for whom an opioid agonist prescription is appropriate, or who is otherwise at risk of opioid overdose) or to a second individual.

The term “solvate,” as used herein, refers to an opioid antagonist described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “subject” as used herein refers to any living individual (preferably human) likely to benefit from treatment with a therapeutically effective amount of nalmefene. In some embodiments, the subject is exposed intentionally or unintentionally to an opioid agonist, such as fentanyl. In additional embodiments, the subject may include, but is not limited to, members of the military, law enforcement, professional security personnel, or personnel providing emergency medical services.

“Therapeutically effective amount” or “therapeutically effective dose”, as used herein means effective to treat an opioid overdose or symptoms thereof caused by exposure of an opioid agonist in a subject. Opioid overdose may be moderate, severe, or even fatal. In some embodiments, a treatment or pharmaceutical formulation will be therapeutically effective to treat an even moderate opioid overdose, wherein a subject is temporarily physically or mentally impaired by an opioid agonist but is in no danger of death. In some embodiments, a treatment or formulation will be therapeutically effective to treat a severe overdose, wherein a subject is in danger of death. In some embodiments, a treatment or formulation will be therapeutically effective to prevent fatal overdose.

“Exposure” as used herein refers to an actual or suspected contact between the subject and an opioid agonist. Actual exposure refers to exposure that in fact occurs whether known or unknown. Suspected exposure refers to any level of expected possibility of being exposed to an opioid.

“Unintentional exposure to an opioid agonist by a subject” as used herein means that the exposure to an opioid agonist is not voluntary and/or intended to self-intoxicate but occurs as part of the subject's activities in proximity to, or potential proximity to, the opioid agonist. For example, unintentional exposure of opioid naïve subjects who take stimulants (e.g., cocaine, methamphetamine) or cannabis contaminated with fentanyl. See Bazazi et al. Overdose from Unintentional Fentanyl Use when Intending to Use a Non-opioid Substance: An Analysis of Medically Attended Opioid Overdose Events. J Urban Health. 2024 April; 101(2):245-251. Other examples include workers acting in capacity as law enforcement, inspectors, public health field agents, who may face the risk of unintentional exposure during activities such as cleaning or performing inspections or investigations for and/or handling opioid agonists or materials that are associated with opioid agonists, such as laboratory equipment, containers, needles, pipes or other objects.

Additionally, military, law enforcement, or security personnel may face incidental exposure to opioid agonist when a third party deliberately delivers an opioid agonist to incapacitate said personnel, for example as a gas. Accordingly, incidental exposure to opioid agonist includes, for example, exposure by inhalation to aerosolized opioid agonist and incidental transdermal exposure to opioid agonist.

“Aerosolized opioid agonist” as used herein means that the opioid agonist is delivered through the air to a subject such as, a gas, mist, or fine powder. It does not include opioid agonist in powder form that is voluntarily inhaled (e.g., snorted) by a subject. An example of an aerosolized opioid agonist is fentanyl (or a derivative thereof) administered through the ventilation system of a building to anaesthetize, incapacitate, or kill the occupants.

The term “untrained individual” refers to an individual administering an opioid antagonist using a device described herein, wherein the individual is not a healthcare professional and has received either no or minimal training in the use of the device.

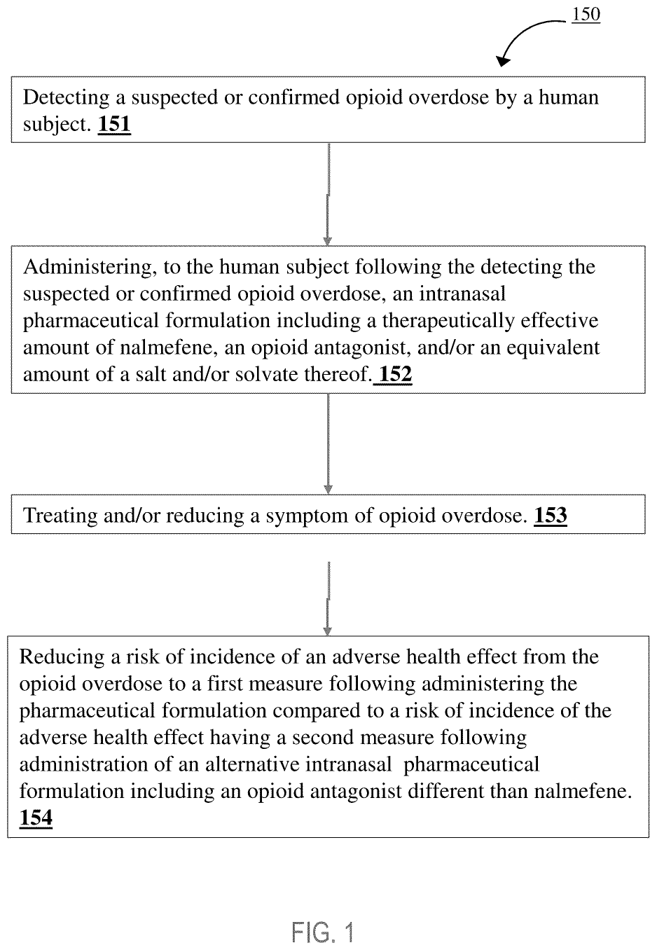

FIG. 1 is a flowchart illustrating a method 150 of treating opioid overdose in a subject, as disclosed herein, according to an embodiment. As shown in FIG. 1, method 150 includes step 151 of detecting a suspected or confirmed opioid overdose and/or exposure of a human subject. In some instances, the detecting can be by an individual who detects a subject who is or will be in need of treatment of opioid overdose (e.g., bystander or medical professional who detects opioid overdose in a drug user). In some instances, the detecting can be detecting a past opioid overdose after the opioid overdose has occurred (e.g., post-hoc identification of opioid overdose by first responders). In some instances, the detecting can include determining and/or inferring a source or identity of the opioid and/or amount of the opioid that caused the overdose (e.g., if it is a synthetic opioid like fentanyl or carfentanil and the estimated amount of opioid that to which the subject may have been exposed).

In some instances, the detecting of past opioid overdose can be performed by any individual or entity including first responder, medical or healthcare professional, personnel providing emergency assistance services, law enforcement personnel, military personnel, security personnel, any bystander, or trained or untrained member of the public. In some instances, the detecting of imminent opioid overdose can be based on a situational prediction of an imminent opioid overdose (e.g., prediction of a potential exposure by a first response in a drug raid) from firsthand or secondhand knowledge about the circumstances of the exposure. In some instances, the detecting of past opioid overdose can be based on any information about the circumstances and/or suitable metrics or parameters symptomatic of opioid overdose. For example, the detecting can be based on visible and/or audible signs of opioid overdose or suspected opioid overdose including unresponsiveness and/or loss of consciousness defined by lack of response to stimuli such as shouting, shaking, or rubbing knuckles on sternum; slow, erratic, or complete cessation of breathing; slow, erratic, or cessation of a pulse; deep snoring or choking/gurgling sounds; indications of reduced blood circulation and/or blood oxygenation; signs of cyanosis and/or blue or purple fingernails or lips; pale and/or clammy face; slack or limp muscle tone; contracted pupils; and vomiting.

Following the detecting of an imminent or past opioid overdose at 151, at step 152, the method 150 includes administering, to the human subject following the detecting of the suspected or confirmed opioid overdose an intranasal pharmaceutical formulation including a therapeutically effective amount of nalmefene, an opioid antagonist, and/or an equivalent amount of a salt and/or solvate thereof.

The administering can be done using any known suitable device and/or system configured to be operable in the field as an immediate response. For example, the administering can be done using a pre-loaded and primed intranasal spray configured to deliver a pre-determined dosing or amount of the intranasal pharmaceutical formulation. The dosing can be any appropriate amount based on prior research and studies, and/or based on the detecting the opioid overdose.

The method 150 includes, at step 153, treating and/or reducing a symptom of opioid overdose. The symptom can be one or more effects disclosed herein including respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, vomiting, brain hypoxia, cardiac arrest, and/or the like.

The method 150 includes, at step 154, reducing a risk of an adverse health effect resulting from the opioid overdose following administration of the pharmaceutical formulation compared to a risk of incidence of the adverse health effect having a second measure following administration of an alternative intranasal pharmaceutical formulation including an opioid antagonist different than nalmefene, such as naloxone.

Indeed, in some instances, the alternative intranasal pharmaceutical formulation can include intranasal naloxone, an alternative opioid antagonist, compared to intranasal nalmefene. As described, the adverse health effect can include at least one of respiratory depression, cardiac arrest, brain hypoxia, and death. As an example, the risk of incidence of an adverse health effect following administering the pharmaceutical formulation including intranasal nalmefene and the risk of incidence of the adverse health effect following administration of an alternative intranasal pharmaceutical formulation including intranasal naloxone can be calculated using predictions from simulations conducted using the Laffont Model 100 described herein.

In some instances, detecting the opioid overdose and/or reducing the predicted first risk of incidence of the adverse health effect from the opioid overdose to a first measure following the administration of the first intranasal pharmaceutical formulation can include calculating a minute ventilation association with the subject, and determining a decrease in minute ventilation below an identified threshold value (measure of magnitude of respiratory depression) for detection of opioid overdose and subsequent increase in the minute ventilation to reach baseline value associated with that subject (measure of effectiveness of reversal). In some instances, the identified threshold value for decrease in minute ventilation for detecting overdose can be 40% of baseline, wherein a reduction below this identified threshold compared to a baseline minute ventilation associated with that subject can be indicative of an unsafe level of respiratory depression suffered by the subject.

In some instances, the detecting can be associated with a time of identification of the minute ventilation of the subject reducing below the identified threshold as described above. In some instances, administration of the intranasal pharmaceutical formulation can be subsequent to the detecting at what is referred to as “delivery time” that is less than or equal to 1 minute from the detecting when the minute ventilation reduces below the identified threshold. In some instances, the administering the intranasal pharmaceutical formulation can be at the “delivery time” that is up to 3 minutes from the time of detecting when the minute ventilation reduces below the identified threshold.

### Example Studies

Implementation of the present disclosure is provided by way of the following Examples. The Examples serve to illustrate the methods and/or formulations disclosed herein without being limiting in nature.

### Study I: Reversal of Opioid-Induced Respiratory Depression in Healthy Volunteers: Comparison of Intranasal Nalmefene and Intranasal Naloxone

An open-label, randomized, crossover study was conducted in healthy volunteers to compare the reversal of respiratory depression induced by the opioid remifentanil by delivering an intranasal (IN) pharmaceutical formulation of naloxone hydrochloride (4 mg) or an intranasal (IN) pharmaceutical formulation of 3 mg nalmefene hydrochloride (corresponding to 2.7 mg base) (NCT 04828005). In this study, subjects were administered a hypercapnic gas mixture which produces an elevation in minute ventilation (MV), a result of the ventilatory response to hypercapnia. After initiating the hypercapnic gas mixture and the elevation in minute ventilation, a remifentanil bolus was administered, and an infusion continued for the study duration. To treat remifentanil-induced respiratory depression, subjects were administered either IN naloxone or IN nalmefene 15 min after initiating the remifentanil infusion using methods and/or pharmaceutical formulations disclosed herein, according to an embodiment. MV was monitored. Results showed that both nalmefene and naloxone produced a time-dependent reversal of remifentanil-induced reductions in MV. At the primary endpoint (5 min post administration), treatment using IN pharmaceutical formulation consisting of nalmefene as the active ingredient resulted in increases in MV (5.75 L/min) that were nearly twice that produced by IN pharmaceutical formulation consisting of naloxone (3.01 L/min). The point estimate favored nalmefene, demonstrating non-inferiority and superiority. In this clinical model of opioid-induced respiratory depression, IN pharmaceutical formulation consisting of nalmefene as the active ingredient showed a more rapid onset of reversal or treatment action than IN pharmaceutical formulation consisting of naloxone as the active ingredient, which required 20 min to achieve a comparable reversal of respiratory depression. Although both nalmefene and naloxone were well tolerated by healthy volunteers, the rapid onset of action with delivery of IN pharmaceutical formulation consisting of nalmefene as the active ingredient may prove particularly valuable in an era when over 90% of fatalities are linked to synthetic opioid overdose.

### Methods

Ethical considerations precluded using an unapproved method and/or formulation (at the time of study) as a rescue medication in a field study. However, multiple studies in healthy volunteers have demonstrated hypercapnia-induced increases in minute ventilation (MV), a physiological effect of the ventilatory response to hypercapnia (VRH), is reduced by opioids and reversed by parenteral administration of opioid antagonists. In this study, in an open-label crossover design study with a non-inferiority endpoint, a clinical model of opioid-induced respiratory depression from hypercapnia-induced increases in MV was used to compare a 4 mg dose of IN naloxone hydrochloride to a 2.7 mg dose of IN nalmefene in reversing reductions in MV produced by remifentanil, a synthetic opioid widely used as an anesthetic and analgesic.

The protocol and related documents for this study were reviewed and approved by an institutional review board, WCG IRB (Puyallup, WA). All subjects provided written informed consent prior to participation in this study. The study was conducted at ICON— Early Development Services (Salt Lake City, UT) in accordance with the ethical principles that have their origins in the World Medical Association (WMA) Declaration of Helsinki, adopted by the 18th WMA General Assembly, Helsinki, Finland and subsequent amendments. The study design also complied with ICH E6 Guideline for GCP (European Medicines Agency [EMA]/Committee for Medicinal Products for Human Use [CHMP]/ICH/135/1995) and clinical research guidelines established by the Basic Principles defined in the US 21 CFR Parts 50, 56, and 312. The study was registered (www.clinicaltrials.gov) as NCT 04828005.

### Participant Characteristics

A total of 69 healthy male and female volunteers were randomized to receive study drugs using an open-label, crossover design. The demographics for sex, race, ethnicity, age, weight, height, and body mass index (BMI) are shown in Table 1. To be included in the study, male and female (including females of childbearing potential, non-childbearing potential, or menopausal) volunteers were required to meet age (18-55 years), weight (at least 50 kg), and BMI (18-32 kg/m²) criteria at screening. Participants were required per protocol to be non-opioid dependent but had been exposed to an opioid on at least 1 occasion prior to screening. Screening procedures conducted within 28 days of study initiation included the following: medical history, demographics, eligibility (based on inclusion/exclusion criteria), concomitant medications, physical examination, nasal cavity examination, Mallampati intubation score, alcohol breath test, clinical chemistry, coagulation markers, hematology, infectious disease serology, urinalysis, urine drug, urine cotinine and alcohol toxicology screen, serum pregnancy tests (female subjects), vital signs, and electrocardiogram (ECG). Subjects with a positive screening test for hepatitis B surface antigen (HBsAg), anti-hepatitis C virus (HCV) antibodies, or anti-human immunodeficiency virus (HIV)-1 and -2 antibodies were excluded from the study as were individuals who had donated or received blood or undergone plasma or platelet apheresis within 30 days prior to the scheduled initial dosing. Healthy status was defined by the absence of evidence of any clinically significant active or chronic disease in the opinion of the Investigator following a detailed medical and surgical history, a complete physical examination including vital signs, 12-lead ECG, hematology, blood chemistry, serology, and urinalysis. Enrolled subjects were required per protocol to be free from upper respiratory tract infection within 7 days prior to first dose of study drug and screened both for the ability to tolerate wearing a tight-fitting mask and respond with an increase in MV when exposed to a hypercapnic gas mixture (VRH test). Subjects were also required to have adequate venous access as determined by the Investigator. These subjects were required per protocol to have had no previous or current opioid, alcohol, or other drug dependence (excluding nicotine and caffeine) based on medical history and passed a naloxone challenge test (see Supplemental Materials) at admission (day −1) (“Day minus 1” or one day prior to start of study) to ensure they were not dependent on opioids. Subjects were required per protocol to have not taken either prescribed or over-the-counter (OTC) medications including decongestants, dietary supplements, herbal products, vitamins, or opioid analgesics for pain relief within 14 days prior to the first dose of study drug and throughout the study, nor had they used either OTC or prescription nasal products for 28 days prior to entering the study. During the study, subjects were required per protocol to abstain from consuming poppy seeds or similar opium-derived foods and from grapefruit/grapefruit juice and alcohol 72 h prior to initial dosing with study drugs and for the remainder of the study. Subjects were also required to restrict drinks containing xanthines (e.g., cola, coffee, and tea) to a limit intake of 500 mg/day. Women of childbearing potential enrolled in the study were required per protocol to be using an accepted means of contraception and were willing and able to continue contraception for 90 days after the last administration of study drug. Women using oral contraception were required per protocol to have initiated use at least 2 months prior to screening. Male subjects admitted to the study and their female partner(s) of childbearing potential agreed, per protocol requirements, to use 2 forms of contraception, one of which was a barrier method during the study and for 90 days following the last administration of study drug. Any recently received experimental agents must have been discontinued per protocol requirements at least 30 days or a period equivalent to 5 half-lives of the agent prior to the first dose of study drug, whichever was longer. On the day of clinic admission (day−1) the following procedures were performed to confirm study eligibility: review of medical history, physical examination (including nasal cavity), ECG, vital signs, weight, height, BMJ, urine pregnancy test (female subjects), urine drug screen and cotinine test, alcohol breath test, review of concomitant medications, and a VRH test. The following vital signs criteria had to be met on either day −1 or pre-dose (day 1) with the subject in a semi-recumbent position: systolic blood pressure: 90 to 140 mmHg; diastolic blood pressure 55 to 90 mmHg; heart rate 55 to 100 beats per minute (bpm); and respiratory rate: 10 to 20 respirations per minute (rpm).

TABLE 1
Demographics of the Study Population

| | Category or Statistics | Number (%) |
|---|---|---|
| Sex | Female | 21 (30.4) |
| | Male | 48 (69.6) |
| Race | White | 57 (82.6) |
| | Black or African American | 6 (8.7) |
| | Asian | 2 (2.9) |
| | American Indian or Alaskan Native | 3 (4.3) |
| | Native Hawaiian or Other Pacific | 1 (1.4) |
| | Not Reported | 0 |
| | Unknown | 0 |
| Ethnicity | Hispanic or Latino | 11 (15.9) |
| | Not Hispanic or Latino | 58 (84.1) |
| | Not Reported | 0 |
| | Unknown | 0 |
| Age (years) | Mean | 29.1 |
| | SD | 7.79 |
| | Median | 27.0 |
| | Min, Max | 18, 52 |
| Weight (kg) | Mean | 78.30 |
| | SD | 14.120 |
| | Median | 79.10 |
| | Min, Max | 50.0, 108.5 |
| Height (cm) | Mean | 175.6 |
| | SD | 10.21 |
| | Median | 176.0 |
| | Min, Max | 143, 198 |
| BMI | Mean | 25.27 |
| | SD | 3.316 |
| | Median | 24.90 |
| | Min, Max | 18.1, 31.7 |

Subjects were ineligible for the study if there was a history of any clinically significant disease, severe seasonal or non-seasonal allergies, nasal polyps or any nasal passage abnormality that could interfere with nasal spray administration (including changes in turbinates, chronic rhinorrhea, sinusitis, or excessive sneezing). Subjects were also excluded from the study if there was a known hypersensitivity reaction to plastics or ECG lead adhesive, allergy to opioids, nalmefene, or naloxone or any of the excipients in the test agents, a history of migraines, seizures, or known electroencephalogram (EEG) abnormalities, a history of suicidality based upon clinical history, source documents, or scores on the Columbia-Suicide Severity Rating Scale, or any other condition which, in the opinion of the Investigator, would jeopardize the safety of the subject or impact the validity of the study results.

### Study Design

The randomized, 2-period, 2-treatment crossover study was designed to evaluate the pharmacodynamic effects of IN naloxone compared to IN nalmefene to reverse opioid-induced suppression in MV. The objectives included to demonstrate non-inferiority at the primary endpoint (5 min post administration) of IN nalmefene compared to IN naloxone to reverse remifentanil-induced reductions in MV and to determine the tolerability of IN nalmefene during remifentanil infusion. The study consisted of an outpatient screening visit, an in-clinic treatment phase, and a follow-up phone call. On the days of drug administration (i.e., days 1 and 5), 69 subjects fasted for a period of at least 8 h and received pretreatment with famotidine (20 mg IV), ondansetron (S mg, oral), and sodium citrate (30 mL, oral) 30 min to 1 h prior to remifentanil infusion.

### Drug Administration Protocol

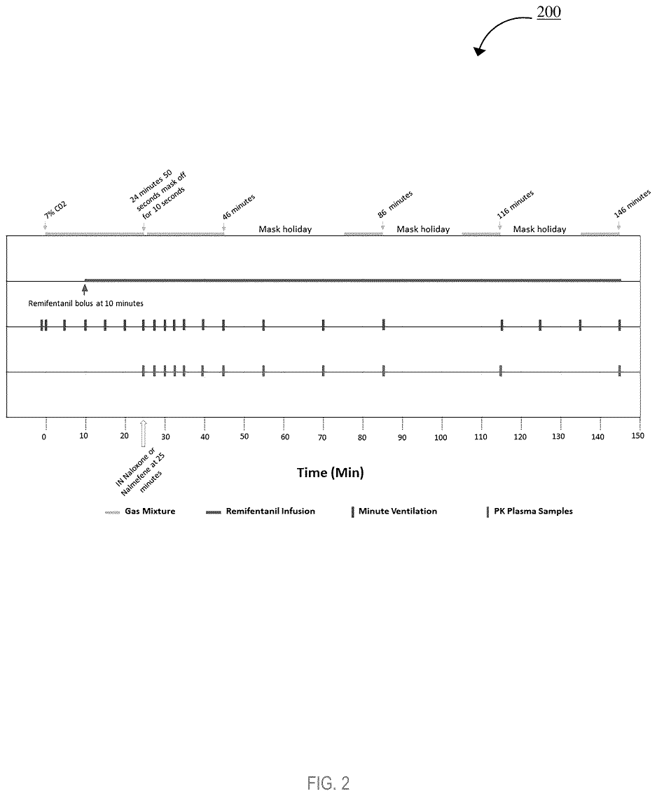

FIG. 2 illustrates a schematic showing the protocol adopted for drug administration for this study. Subjects breathed room air for at least 5 min through a tightly sealed VRI-L face mask while lying on a bed at 45° recumbent to capture baseline MV monitored using the ExSpiron device. Subjects then started breathing a hypercapnic gas mixture (50% O₂, 43% N₂, and 7% CO₂) through the VRH face mask (time 0 min) (FIG. 1). Ten minutes later, subjects received an initial bolus of remifentanil hydrochloride (0.5 μg/kg) followed by an infusion at a rate of 0.175 μg/kg/min. Multiple studies have demonstrated that a remifentanil bolus followed by an infusion produces steady-state blood levels within minutes. At 24 min 50 s, the VRH face mask was removed for approximately 10 s; during this time, subjects were asked to hold their breath while either IN nalmefene (2.7 mg, equivalent to 3 mg of nalmefene HCl) or naloxone HCl (4 mg) was administered 15 min after the start of the remifentanil infusion. The VRH face mask was reapplied, and the remifentanil infusion continued for a further 121 min up to 146 min. The VRH face mask was removed 21 min after IN administration of either nalmefene or naloxone hydrochloride (time 46 min); this and subsequent “mask holidays” (FIG. 1) were introduced to reduce subjects' discomfort and encourage study completion. The VRH face mask was then reapplied for 11 min at time 75, 105, and 135 min, respectively, (i.e., between 50 to 61 min, 80 to 91 min, and 110 to 121 min after IN administration of study drugs) as shown in the protocol schematic in FIG. 2. Of the 69 subjects who received a single dose of either IN nalmefene or IN naloxone, a total of 52 subjects received both treatments, with data collected at the primary endpoint in 50 subjects.

### Bioanalytical Methods

Plasma nalmefene and naloxone concentrations were analyzed using a validated liquid chromatography-tandem mass spectrometry (LC-MS/MS) assay. Plasma samples (0.2 mL) were transferred to a 96-well polypropylene plate followed by 0.025 mL (2.50 ng/mL, each, in methanol/water [1:1]) of the nalmefene-D5 and naloxone-D5 working internal standard solution. The samples were mixed for 60 s, 0.200 mL of each sample was transferred to a support liquid extraction plate (ISOLUTE SLE 200 μL, Biotage). The samples were eluted with 0.8 mL of ethyl acetate/isopropyl alcohol (95:5), and the extracts were evaporated to dryness at 45° C. under a stream of nitrogen. The dried sample was reconstituted with 0.1 mL of 0.1% formic acid in water and stored at 10° C. until analysis.

Analyses were performed using a Waters Acquity UPLC (Waters Corp, Milford, MA) and Sciex API 6500 MS/MS with a TurboIonSpray source (AB Sciex, Toronto, Ontario, Canada) system. Nalmefene and naloxone were eluted with 15% mobile phase A (20 mM ammonium formate pH 2.5 adjusted with formic acid) and 85% mobile phase B (100% acetonitrile) at a target flow rate of 1.00 mL/min through a Zorbax 300SCX 50×3 mm column (Phenomenex, Torrance, CA). Under these conditions, nalmefene and naloxone eluted at approximately 1.36 and 2.15 min, respectively. Nalmefene was quantified by comparing the peak area of m/z 340.3→268.3 to that of nalmefene-D5 (m/z 345.3→273.2). For naloxone, the peak area of the m/z 328.3→212.2 was compared to that of naloxone-D5 (m/z 333.2→217.2). A weighted 1/x² linear least squares regression analysis generated from calibration standards was used to quantify nalmefene and naloxone concentrations. The method was validated over a range of 0.01-10.0 ng/mL as the lower and upper limits of quantitation for both. The calibration curves for nalmefene were linear (r²>0.9951) over the specified concentration range; the interday precision of the calibration curves and quality control samples ranged from 1.3% to 4.6%, and the accuracy ranged between −2.8% and 5.6% during sample analyses. The calibration curves for naloxone were linear (r2>0.9931) over the specified concentration range. The interday precision of the calibration curves and quality control samples ranged from 3.1% to 8.4%, and the accuracy ranged between −5.7% and 3.0% during sample analyses.

### Pharmacokinetic Calculations

Maximum plasma concentration (Cₘₐₓ, ng/mL) and time to Cₘₐₓ (Tₘₐₓ, h) were the observed values. CX min was the value observed at the specified time. Area under the plasma concentration time curve from time zero to 20 min (AUC₀₋₂₀, ng·h/mL) was calculated using the linear trapezoidal method.

### Study Drugs

Nalmefene nasal spray, delivered in an Aptar Unidose device (Louveciennes, France) was manufactured by Summit Biosciences (Lexington, Kentucky). Each device delivered 0.1 mL containing 2.7 mg nalmefene (as 3 mg nalmefene HCl). Naloxone nasal spray (containing 4 mg naloxone HCl) and Ultiva for injection (remifentanil HCl) were sourced by the ICON Pharmacy. The hypercapnic gas mixture (7% CO₂, 43% N₂, and 50% O₂) was supplied by Norco, Inc. (Salt Lake City, UT).

### Estimation of Sample Size and a Non-Inferiority Margin

This study was designed to demonstrate the non-inferiority of IN nalmefene (test agent) compared to IN naloxone (reference agent) to reverse remifentanil induced depression of MV at the primary endpoint, 5 min post administration. In the absence of data examining the effects of IN opioid antagonists on opioid-induced depression in MV, the parameters used to determine sample size were estimated from the available clinical literature. Prior research has reported that following morphine-induced depression of MV, intravenous naloxone (0.4 mg) produced about a 3 L/min increase in MV at 5 min post administration. A non-inferiority margin within 80% of the estimated reversal produced by naloxone (i.e., a difference of approximately 0.6 L/min between treatments based on the prior study) was found not to represent a clinically significant difference. The standard deviation of treatment difference was estimated to be 1.75 L/min based on a study comparing intravenous doses of naloxone and nalmefene to reverse morphine-induced depressions in MV that reported a standard deviation of differences in MV between treatments of 1.26 L/min. Based on these reports, with a sample size of 46 subjects in a crossover design, the coverage probability was 90% that the upper limit of a 2-sided 95% confidence interval (CI) for the difference between the treatments (reference [naloxone] test [nalmefene]) in mean change from the remifentanil-induced nadir for MV would be less than the non-inferiority limit of 0.6 L/min given α=0.025 and a standard deviation of 1.75 L. Interim analyses were conducted for sample size estimation, with sample size based on data generated after 22 and 50 subjects were dosed. Based on the initial estimate of having 46 subjects complete both treatments at the primary endpoint of 5 min post dose, planned enrollment was up to 68 subjects. Statistical analyses of non-inferiority were performed using only subjects who had both treatments and completed data collection at the primary endpoint. The primary endpoint of change in MV after study drug administration was analyzed using the linear model for a 2-treatment, 2-period crossover study. The model included treatment, period, and sequence as fixed effects, and subject nested within sequence as a random effect. The least squares (LS) means and the mean difference (naloxone minus nalmefene) of change in MV and corresponding 2-sided 95% CI were estimated. A total of 69 subjects was included in the intent-to-treat set: 61 received 2.7 mg IN nalmefene and 60 received IN naloxone, respectively; 50 subjects received both treatments and completed the primary endpoint.

### Results

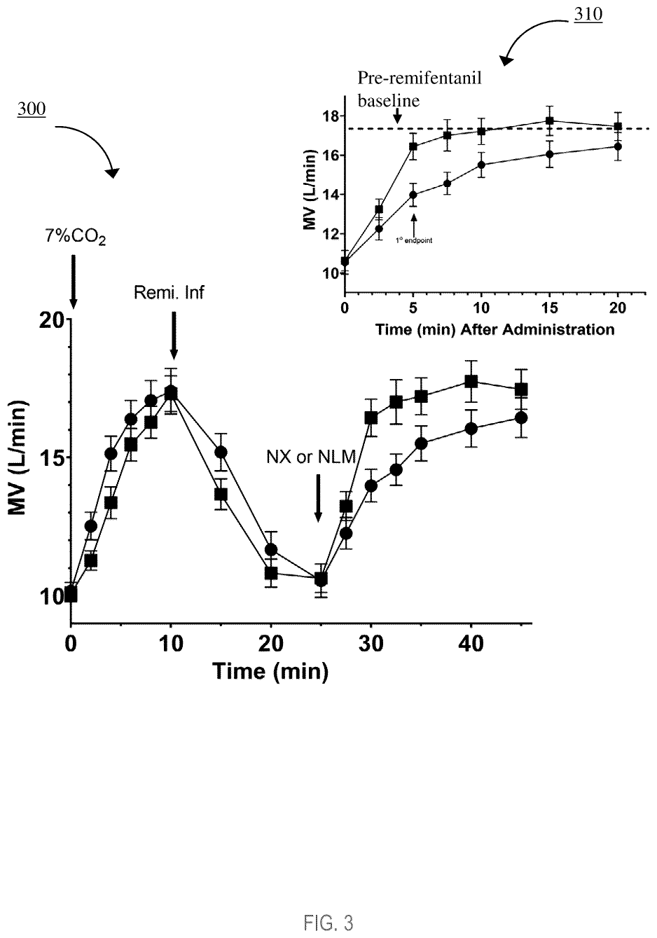

Reversal of Remifentanil-Induced Respiratory Depression by Opioid Antagonists was observed using IN pharmaceutical formulations and methods disclosed herein, according to an embodiment. FIG. 3 illustrates MV from initiation of the hypercapnic gas mixture through the first mask holiday. Plot 300 in FIG. 3 shows changes in MV through the first mask holiday (T+46 in protocol in FIG. 2). Trace with square markers represents data corresponding to intervention with IN nalmefene and trace with circle markers represents data to corresponding to intervention with IN naloxone. The time point of 7% CO2, initiation of the hypercapnic gas mixture is indicated by the downward arrow (T=0). The time point of administration of a remifentanil bolus followed by an infusion is indicated by the downward arrow at T=10 labeled “Remi.Inf.” The time point of administration of intranasal pharmaceutical formulation comprising at least one pharmaceutically acceptable carrier and consisting of a therapeutically effective amount of nalmefene, NLM, or naloxone, NX, is indicated by the label “NX or NLM” administered at T=25 marker by the downward arrow.

Plot 310 in the inset in FIG. 2 shows the speed of onset of reversal of respiratory depression by IN naloxone and IN nalmefene. Naloxone or nalmefene was administered at time “0” (corresponding to T=25 on the lower panel). The primary endpoint (5 min post administration corresponding to T=30 on the lower panel) is indicated by the upward arrow. The dotted horizontal line represents the pooled mean MV of subjects following 10 min of exposure to the hypercapnic gas mixture. In plot 310 again, the trace with square markers corresponds to data from administration of IN nalmefene, and the trace with circle markers corresponds to data from administration of IN naloxone. MV data was based on the average of a 60 s measurement (12×5 s epochs) taken 30 s on either side of the specified time point (e.g., MV at 15 min reflects data collected between 14 min 30 s and 15 min 30 s). Data represent the MV (X±SEM) in L/min of 61-65 subjects.

As shown in FIG. 3, remifentanil reduced the increase in MV produced by a 10 min exposure to the hypercapnic gas mixture in a time-dependent manner. Both IN naloxone and IN nalmefene reversed remifentanil-induced reductions in MV during the first 20 min post administration. The reversal was apparent for both antagonists as early as 2.5 min post administration, the earliest time point measured (as shown in plots 300 and 310). The reversal by IN nalmefene was more rapid, reaching approximately 95% of the pre-remifentanil baseline within 5 min with an apparent complete reversal maintained through the first 20 min post administration (see plots 300 and 310 in FIG. 3). The reversal produced by IN naloxone was more gradual, with 20 min required to restore MV to the values observed 5 min after IN nalmefene administration (plots 300 and 310 in FIG. 3).

The difference in rapidity of reversal between IN naloxone and IN nalmefene was apparent whether the data were expressed as either the absolute MV (plots 300 and 310 in FIG. 3) or as the mean change in MV for each subject between 2.5 and 20 min post administration and their remifentanil-nadir baseline (Table 2).

TABLE 2
Reversal of Remifentanil-Induced Reduction in MV by Naloxone
and Nalmefene: Effects of Time and Mask Holidays

| | Naloxone ΔMV | Nalmefene ΔMV |
|---|---|---|
| Time | (L/min) | (L/min) |
| 2.5 | 1.71 ± 0.53 | 2.55 ± 0.38 |
| 5 | 3.43 ± 0.61 | 5.74 ± 0.62 |
| 7.5 | 4.01 ± 0.63 | 6.38 ± 0.71 |
| 10 | 4.96 ± 0.69 | 6.58 ± 0.66 |
| 15 | 5.55 ± 0.64 | 7.11 ± 0.77 |
| 20 | 5.93 ± 0.68 | 6.79 ± 0.71 |
| 21-50 (Mask holiday) | | |
| 60 | 2.52 ± 0.58 | 2.92 ± 0.56 |
| 61-80 (Mask holiday) | | |
| 90 | 2.14 ± 0.62 | 2.71 ± 0.59 |
| 91-110 (Mask holiday) | | |
| 120 | 2.02 ± 0.52 | 2.88 ± 0.60 |

Following each of the 3 mask holidays and reapplication of the hypercapnic gas mixture for 10 min in the protocol (see FIG. 2)), MV in nalmefene-treated subjects was consistently, albeit only slightly higher than the corresponding values following naloxone administration (Table 2). However, the changes in MV following these mask holidays were substantially lower for both nalmefene and naloxone than those observed during the first 20 min following drug administration (i.e., prior to the first mask holiday) (Table 2).

### Primary Endpoint Analysis

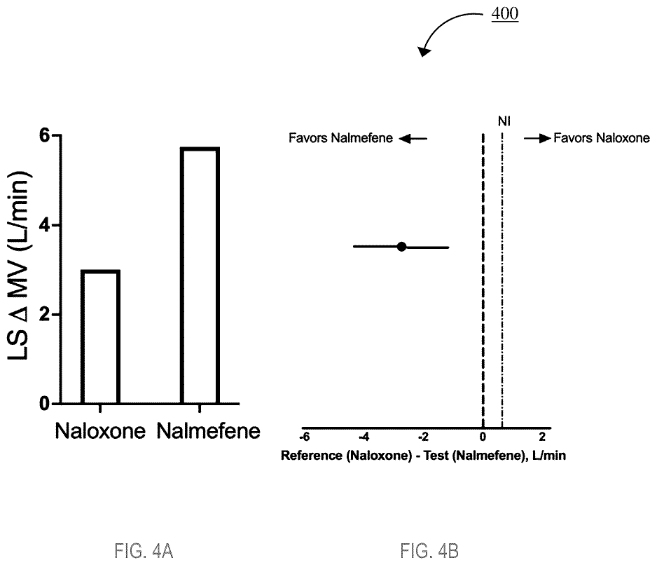

FIGS. 4A and 4B show plots illustrating the reversal of remifentanil-induced respiratory depression by IN naloxone and IN nalmefene, using methods formulations and/or methods disclosed herein, according to an embodiment, as evaluated through primary endpoint analysis. This study was designed to demonstrate, per regulatory guidance, non-inferiority at the pre-specified primary endpoint (5 min post administration) using naloxone as the reference agent and nalmefene as the test agent. The analysis was conducted according to Food and Drug Administration (FDA) guidance on non-inferiority trials (FDA, 2016; https://www.fda.gov/media/78504/download).

FIG. 4A shows least squares (LS) mean change in minute ventilation (ΔMV) (i.e., mean recovery in minute ventilation) from the remifentanil-induced nadir to 5 min post-drug administration. Per the protocol, only subjects from the intent-to-treat (ITT) population who received both treatments and completed data collection for at least 5 min post dose (n=50) were included in the analyses. Analyses were performed using a linear mixed effects model with treatment, period, and sequence as fixed effects and subject nested within sequence as a random effect. The LS mean changes in MV from the remifentanil-induced nadir to 5 min post dose for naloxone and nalmefene were 3.01 and 5.74 L/min, respectively. The difference in mean change between naloxone and nalmefene (2.73 L/min) was statistically significantly (P<0.0009).

FIG. 4B shows non-inferiority analysis of reference (IN naloxone) versus test (IN nalmefene) agent at the primary endpoint, 5 min post-drug administration, identified by the upward arrow in plot 310 in FIG. 3. The circle marker represents the least squares difference, −2.73 L/min, (with 95% confidence intervals (CI)) from the remifentanil-induced nadir to 5 min following IN administration of naloxone and nalmefene. The vertical line labeled NI (non-inferiority) is 20% of the LS mean change for naloxone (0.602 L/min), the pre-established non-inferiority margin. The dashed vertical line (at X=0) represents point of no difference between treatments.

At the primary endpoint of 5 min post administration the least squares (IS) mean difference in MV from the remifentanil-nadir baseline was 5.74 and 3.01 L/min following nalmefene and naloxone, respectively (FIG. 4A). In this study, non-inferiority would be established if the upper limit of the 95% CI for nalmefene was less than the pre-specified margin, 20% of the mean change in MV produced by naloxone (0.6022 L/min). Non-inferiority was established with the LS mean difference in MV between treatments (−2.73 L/min) that was statistically significant (P<0.0009), with the upper limit of the 95% CI (−1.17 L/min) favoring nalmefene (FIG. 4B).

Based on the FDA guidance for non-inferiority trials, nalmefene was found both non-inferior to and superior to naloxone in its treatment or reversal of remifentanil-induced respiratory depression as evidenced by recovery from opioid induced depression of hypercapnia-induced raise in minute ventilation.

These results were further confirmed following a sensitivity analysis that excludes subjects for which the change in MV at 5 min was more than 15 interquartile range (IQR) below the first quartile (Q1) or more than 1.5 IQR above the third quartile (Q3). In this analysis (n=48 subjects), the LS mean difference between naloxone and nalmefene was 2.51 L/min (95% CI: 3.73, 1.28 L/min).

### Secondary and Exploratory Endpoints

Secondary endpoints were ordered on a hierarchical basis beginning with a comparison of the maximum change in MV from the remifentanil-induced nadir (https://www.clinicaltrials.gov/study/NCT04828005). No statistically significant difference in the maximum change in MV was observed (Table 2), hence no other secondary endpoints were analyzed.

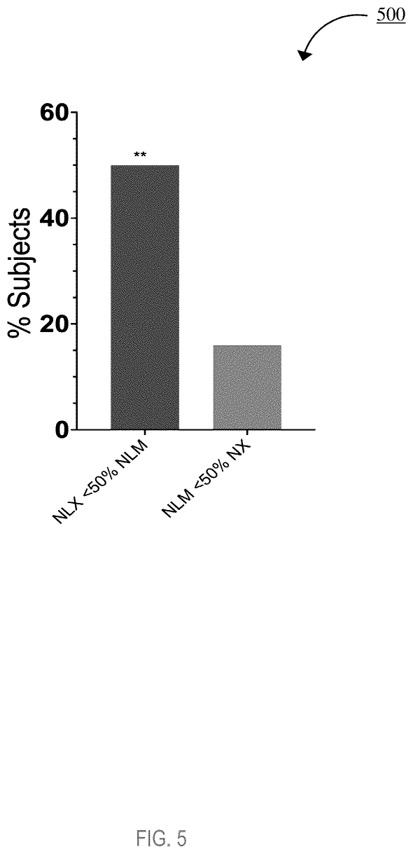

In an exploratory analysis of the primary endpoint, the proportion of subjects who had a change in MV of less than 50% of the nalmefene response following naloxone was compared to the proportion of subjects who had less than 50% of the naloxone response following nalmefene. The results are illustrated in FIG. 5 as a proportion of subjects with “extreme responses” at the primary endpoint.

FIG. 5 shows a comparison between the proportion of subjects with a change in minute ventilation (MV) following naloxone that was less than 50% of their change in MV following nalmefene at 5 min post administration was compared to the proportion of subjects with a change in minute ventilation after nalmefene that was less than 50% of their change in minute ventilation after naloxone. Only subjects who received both treatments and completed data collection for at least 5 min post dose (n=50) were included in the analyses. Twenty-five subjects had an MV after naloxone that was <50% of their response to nalmefene. Out of this set, 8 subjects had a change in MV after nalmefene that was <50% of their response to naloxone, **P<0.0003, under Chi-square test. As shown in FIG. 5, there was a statistically significant difference (P<0.0003, Chi-square analysis) in the percent of subjects (50%) who had a change in MV less than 50% following naloxone compared to nalmefene compared to 16% of subjects whose response was less than 50% following nalmefene compared to naloxone.

### Pharmacokinetics of IN Naloxone and Nalmefene

At the indicated times for blood sample collection in the protocol in FIG. 2, approximately 4 mL of blood was collected via an indwelling IV catheter into dipotassium ethylenediaminetetracetic acid (K2EDTA) containing tubes for separation of plasma. Naloxone and nalmefene were analyzed using a liquid chromatography-tandem mass spectrometry method as described in Methods above. Maximum plasma concentration (Cmax, ng/mL) and time to Cmax (Tmax, h) were observed. CXmin was the value observed at predefined specified times. Area under the plasma concentration time curve from time zero to 20 min (AUC0-20, ng·h/mL) was calculated using the linear trapezoidal method. Observed values are shown in Table 3 alongside comparator values for IN Naloxone (NX Comp,) and comparator values for IN Nalmefene (NLM Comp.).

Naloxone comparator (NX Comp.) data was obtained from a study by Krieter et al. including 29 subjects (see, Krieter P, et al. Pharmacokinetic properties and human use characteristics of an FDA approved intranasal naloxone product for the treatment of opioid overdose. J Clin Pharmacol. 2016; 56(10):1243-1253.). The Cₘₐₓ and Tₘₐₓ values for IN NX Comp. were consistent with concurrent reports using 4 mg IN naloxone in healthy volunteers.

Nalmefene comparator (NLM Comp.) data was obtained from a study by Crystal et al. including 66 subjects (see, Crystal R, Ellison M, Purdon C, Skolnick P. Pharmacokinetic properties of an FDA-approved intranasal nalmefene formulation for the treatment of opioid overdose. Clin Pharmacol Drug Dev. 2024; 13(1):58-69). The Cₘₐₓ, Tₘₐₓ, and Cₜᵢₘₑ and AUC0-20 values for the comparator nalmefene trial are also consistent with a second independent trial in a population (n=24) of healthy volunteers described in this study. Markers: + indicates P<0.0005 compared to corresponding nalmefene value; * indicates P<0.001; **, P<0.0001 compared to corresponding values reported in healthy volunteers.

Plasma concentrations of naloxone and nalmefene between 2.5 and 120 min post-drug administration are illustrated in FIG. 6. Notably, the results in Study I were impacted by study conditions like subjects breathing a hypercapnic gas mixture through a face mask. The comparator data were obtained from subjects breathing room air. The differences in breathing conditions therefore, resulted in lower exposure to NLM in Study I compared to comparator data (i.e. absorption was impaired in Study I with a 50% lower Cₘₐₓ). Of note this was only the case for intranasal nalmefene as the nalmefene formulation used an absorption enhancer DDM, the action of which was blunted by breathing a hypercapnic gas mixture.

Values plotted in FIG. 6 represent the X+SEM of 59-61 subjects. Trace with square markers represents data from administration of nalmefene and trace with circle markers represents data from administration of naloxone. Table 3 shows effects of the study conditions on the pharmacokinetics, comparing healthy volunteers exposed to the experimental conditions described in this study to comparator studies in healthy volunteers administered intranasal either naloxone or nalmefene. Plasma concentrations of naloxone and nalmefene were taken from data indicated in FIG. 5 and represent the mean of 59-61 subjects. Values are the mean (% CV) except Tₘₐₓ which is the median (range).

Maximum plasma concentrations (Cₘₐₓ) of naloxone and nalmefene were 5.8 and 6.8 ng/mL, respectively, with corresponding Tₘₐₓ values of 45 and 30 min, respectively (Table 3). Prior to the first mask holiday, nalmefene exposure (AUC0-20 min) was approximately 54% higher (P<0.0005) than naloxone (Table 3). Plasma concentrations of nalmefene remained higher than naloxone through the 2-h sampling period (Table 3).

TABLE 3
Effects of Study Conditions on the Pharmacokinetics of Intranasal Naloxone and Nalmefene

| Parameter | NX | | NX Comp. | | NLM | | NLM Comp. | |
|---|---|---|---|---|---|---|---|---|
| Cₘₐₓ (ng/mL) | 5.8 | (49.1) | 5.3 | (44.6) | 6.8 | (40.0)** | 12.2 | (55.2) |
| Tₘₐₓ (h) | 0.75 | (0.13, 2.0) | 0.5 | (0.2, 1.0) | 0.5 | (0.04-2.00) | 0.25 | (0.0833, 2.00) |
| C5 min (ng/mL) | 1.71 | (271) | 1.48 | (117) | 2.66 | (131) | 4.43 | (109) |
| C20 min (ng/mL) | 3.73 | (73.3) | 4.53 | (50.1) | 5.37 | (49.1) | 8.23 | (48.9) |
| C60 min (ng/mL) | 3.92 | (38.1) | 3.28 | (37.1) | 4.92 | (30.8) | 4.64 | (21.5) |
| C120 min (ng/mL) | 2.17 | (38.9) | 1.53 | (51.3) | 3.78 | (27.0) | 4.03 | (19.4) |
| AUC0-20(ng h/mL) | 0.765 | (104)⁺ | 0.886 | (60.3) | 1.18 | (59.3)* | 2.34 | (63.5) |

### Safety and Tolerability

A total of 131 treatment emergent adverse events (TEAEs) were reported by 52 (86.7%) subjects following administration of IN naloxone and 172 TEAEs were reported by 56 (91.8%) subjects following administration of IN nalmefene (Table 4). There were no SAEs, and the majority of subjects reported TEAEs that were either mild (Grade 1; 71%) or moderate (Grade 2; 26.1%) with no significant differences on days subjects received naloxone and nalmefene.

A total of 9 (13.0%) subjects receiving either naloxone or nalmefene discontinued the study due to an AE. Five of these subjects (8.3%) receiving naloxone experienced 4 TEAEs of hypercapnia and 1 TEAE of hypoxia, while 4 (6.6%) subjects receiving nalmefene hydrochloride reported 2 TEAEs of headache and 2 experienced TEAEs of hypercapnia. All TEAEs resolved following discontinuation of the hypercapnic gas mixture and remifentanil infusion.

Overall, nervous system (79.7%) and gastrointestinal disorders (66.7%) were the most frequently reported TEAEs. Among nervous system associated TEAEs, headache was most frequently reported by 35 (58.3%) and 36 (59.0%) of the subjects receiving naloxone and nalmefene, respectively, followed by dizziness, reported by 13 (21.7%) and 11 (18.7%) of subjects, respectively, with dysgeusia and somnolence reported less frequently (Table 4).

TABLE 4
Summary of Treatment Emergent Adverse Events (TEAEs)
by System Organ Class and Preferred Term

| | Naloxone | | Naloxone | | Overall | |
|---|---|---|---|---|---|---|
| System Organ | (N = 60) | | (N = 61) | | (N = 69) | |
| Class Preferred Term | n (%) | | n (%)) | | n (%) | |
| Total subjects with at least | 52 | (86.7) | 56 | (91.8) | 67 | (97.1) |
| one TEAE | | | | | | |
| Gastrointestinal disorders | 26 | (43.3) | 31 | (50.8) | 46 | (66.7) |
| Nausea | 25 | (41.7) | 24 | (39.3) | 39 | (56.5) |
| Vomiting | 14 | (23.3) | 7 | (11.5) | 19 | (27.5) |
| Dry mouth | 0 | | 4 | (6.6) | 4 | (5.8) |
| General disorders and | 3 | (5.0) | 12 | (19.7) | 15 | (21.7) |
| administration site conditions | | | | | | |
| Fatigue | 2 | (3.3) | 6 | (9.8) | 8 | (11.6) |
| Nervous system disorders | 41 | (68.3) | 43 | (70.5) | 55 | (79.7) |
| Headache | 35 | (58.3) | 36 | (59.0) | 48 | (69.6) |
| Dizziness | 13 | (21.7) | 11 | (18.0) | 21 | (30.4) |
| Dysgeusia | 5 | (8.3) | 2 | (3.3) | 6 | (8.7) |
| Somnolence | 4 | (6.7) | 2 | (3.3) | 6 | (8.7) |
| Psychiatric disorders | 4 | (6.7) | 16 | (26.2) | 19 | (27.5) |
| Anxiety | 4 | (6.7) | 10 | (16.4) | 13 | (18.8) |
| Respiratory, thoracic, and | 7 | (11.7) | 17 | (27.9) | 22 | (31.9) |
| mediastinal | | | | | | |
| disorders | | | | | | |
| Hypercapnia | 4 | (6.7) | 2 | (3.3) | 6 | (8.7) |
| Nasal discomfort | 0 | | 5 | (8.2) | 5 | (7.2) |
| Oropharyngeal pain | 2 | (3.3) | 3 | (4.9) | 4 | (5.8) |
| Skin and subcutaneous tissue | 2 | (3.3) | 8 | (13.1) | 9 | (13.0) |
| disorders | | | | | | |
| Hyperhidrosis | 1 | (1.7) | 4 | (6.6) | 5 | (7.2) |
| Pruritus | 1 | (1.7) | 3 | (4.9) | 4 | (5.8) |
| Vascular disorders | 7 | (11.7) | 15 | (24.6) | 19 | (27.5) |
| Hot flush | 7 | (11.7) | 14 | (23.0) | 18 | (26.1) |

Nausea was the most frequent gastrointestinal-related TEALA (56.5% of subjects), with 25 (41.7%) and 24 (39.3) subjects reporting this after naloxone and nalmefene, respectively. Vomiting was reported by 19 (27.5%) subjects, 14 (23.3%) in the naloxone group and 7 (11.5%) in the nalmefene group. Other TEAEs noted were anxiety (18.8% of subjects), with 4 (6.7%) in the naloxone group and 10 (16.4%) in the nalmefene group, and hot flush, noted in 26.1% of subjects, 7 (11.7%) noted following naloxone, 14 (23%) following nalmefene, respectively. Nasal discomfort was reported in 7.2% of subjects (all in the nalmefene group) and oropharyngeal pain in 5.8% of subjects (2 in the naloxone group and 3 in the nalmefene group, respectively).

### Conclusion

The approval of the first IN naloxone product for the treatment of opioid overdose in a community setting was based on a pharmacokinetic endpoint: achieving an onset of action based on exposure equal to or greater than a previously approved parenteral dose. It remains a priority to demonstrate adequate exposure during the early phase (e.g., 2.5 minutes after opioid dosing resulting in opioid overdose) following an attempted rescue or intervention.

Pharmacokinetic studies with IN pharmaceutical formulation of nalmefene (2.7 mg) demonstrated significantly higher exposures than a previously approved parenteral (1 mg IM) dose, including at 2.5 min after dosing, the earliest time point sampled. In addition to this compelling pharmacokinetic data, the FDA requested a pharmacodynamic study to demonstrate that the speed of onset of IN nalmefene was as rapid as an approved naloxone product used in a community setting.

This study establishes that based on the FDA guidance for non-inferiority trials, IN pharmaceutical formulation of nalmefene is both non-inferior to and superior to IN pharmaceutical formulation of naloxone in its treatment or reversal of remifentanil-induced respiratory depression as evidenced by recovery from opioid induced depression of hypercapnia-induced raise in minute ventilation. The study uses a clinical model of opioid-induced respiratory depression from hypercapnia-induced increases in MV to compare a 4 mg dose of IN naloxone hydrochloride to a 2.7 mg dose of IN nalmefene in reversing reductions in MV produced by remifentanil, a synthetic opioid widely used as an anesthetic and analgesic.

Remifentanil was used in this study because steady-state concentrations of this widely used opioid are achieved within minutes by a bolus dose followed by continuous infusion and inactivation by plasma and tissue esterases to eliminate subject-to-subject differences in hepatic metabolism. The dose of remifentanil was well within the recommended ranges for anesthesia and post-operative analgesia (https://www.accessdata.fda.gov/drugsatfda_docs/label/2016/020630s016lbl.pdf); subjects and subjects were responsive to both tactile stimulation and voice commands throughout the study. Under these conditions, remifentanil infusion produced a rapid and robust suppression of MV (FIG. 3) consistent with the reported sensitivity of hypercapnia-induced increases in MV to opioids.

Using the change in MV as a measure of the speed of onset, the objective of the current study was to demonstrate that nalmefene-mediated changes in MV were at least as large as those produced by naloxone. Both naloxone and nalmefene produced a time-dependent reversal of opioid-induced respiratory depression during the first 20 min after administration (FIG. 3). However, respiratory drive was restored to approximately 95% of the pre-remifentanil baseline 5 min following nalmefene treatment, while naloxone required 20 min to restore MV to a comparable level (FIG. 3).

Non-inferiority was demonstrated in subjects who completed the primary endpoint in both arms of this crossover study (FIGS. 4A and 4B) with the difference in MV favoring nalmefene (2.73 L/min; P<0.0009). Moreover, based on the FDA guidance for non-inferiority trials (https://www.fda.gov/media/78504/download) the upper bound of the 95% confidence interval of this difference (1.17 L/min) demonstrated both superiority and non-inferiority. Consistent with the results of the non-inferiority analysis, 25 subjects (i.e., 50% of individuals with data collected in both arms at the primary endpoint) had a change in MV following naloxone that was less than 50% of their response to nalmefene (FIG. 5). By comparison, nalmefene produced a change in MV in 8 subjects (16%) that was less than 50% of their response following naloxone (P<0.0003). While this model is sufficiently sensitive to demonstrate a difference in the speed of reversal between IN naloxone and IN nalmefene, the study conditions employed, including the dose of remifentanil, create a ceiling effect which limits inferences about the relative efficacy of these doses of naloxone and nalmefene.

The mask holidays introduced to reduce subject discomfort and encourage study completion resulted in an apparent blunting of the response to both opioid antagonists (Table 2) despite plasma concentrations equal to or greater than measured during the first 20 min post administration (FIG. 5, Table 3). Further studies may further investigate the effects of mask holidays.

The data presented in this study indicated a more rapid onset of action with IN nalmefene compared to IN naloxone in this clinical model of opioid-induced respiratory depression (FIGS. 3, 4A, and 4B). Nonetheless, previous pharmacokinetic data in normal volunteers indicate the experimental conditions used in the current study underestimate the speed of onset for nalmefene. Thus, in healthy volunteers who were not exposed to a hypercapnic gas mixture both nalmefene exposure during the first 20 min post dosing (AUC₀₋₂₀) and Cₘₐₓ are significantly higher, and the Tₘₐₓ was achieved more rapidly than in the current study (Table 3).

Exposure to a hypercapnic gas mixture has a drying effect on the nasal mucosa (Lynn Webster, personal observation) which may have blunted the effect of the nasal absorption enhancer, dodecylmaltoside (DDM), included in this formulation. DDM increases the absorption of both small molecules and peptides by transiently opening tight junctions between cells in the nasal epithelium and has been demonstrated to both increase the Cₘₐₓ and dramatically reduce the Tₘₐₓ of nalmefene. Consistent with this hypothesis, the most prominent differences in the pharmacokinetic properties of nasal nalmefene between previous studies in healthy volunteers and the current study were observed during the first 20 min post administration, while plasma concentrations at 60 and 120 min were similar (Table 3).

While the introduction of both mask holidays and a remifentanil infusion are also confounding factors, this hypothesis can be tested by a study comparing the pharmacokinetic profile of nalmefene nasal spray in volunteers wearing a tight-fitting mask and breathing either room air or the hypercapnic gas mixture.

The present study conditions also affect the pharmacokinetic profile of naloxone, increasing the Tₘₐₓ from 30 to 45 min, while exposure during the first 20 min was similar to data obtained in healthy volunteers 19 who were not exposed to a hypercapnic gas mixture (Table 3). Although naloxone nasal spray does not contain DDM, a dry nasal environment could also delay its absorption, manifested here as an increased Tₘₐₓ. Paradoxically, remifentanil infusion has been reported to increase exposure to IN naloxone in healthy volunteers. This effect was readily observed 30-90 min after IN administration and was hypothesized to result from remifentanil reducing the pre-systemic metabolism of naloxone swallowed during IN administration. In the present study plasma concentrations of naloxone at 60- and 120-min post dosing were not statistically significantly elevated compared to historical data in healthy volunteers (Table 3).

A high incidence of nausea, vomiting, headache, and dizziness was noted in subjects administered both IN naloxone and nalmefene (Table 4). The profile of adverse events in the current study resembles that reported following remifentanil (e.g., nausea [44%], vomiting [33%], and headache [18%]) administered in monitored anesthesia care studies (https://www.accessdata.fda.gov/drugsatfda_docs/label/2016/020630s016lbl.pdf). Thus, the experimental conditions of the current study (an opioid infusion and intermittent exposure to a hypercapnic gas mixture) may explain the differences in TEAE profiles for both IN naloxone and IN nalmefene between this and previous studies conducted in healthy volunteers.

In summary, this model of opioid-induced respiratory depression was sufficiently sensitive to differentiate the speed of onset of 2 opioid receptor antagonists at the primary endpoint. Furthermore, integrating these data with other translational models of overdose may provide important insights into the utility of nalmefene nasal spray for the treatment of overdose in the synthetic opioid era.

### Study II: Development and Implementation of a Translational Model Assessing the Impact of Synthetic Opioid Overdose/Exposure and Comparison of IN Naloxone and IN Nalmefene on Treating Opioid Induced Respiratory Depression and Cardiac Arrest

As the initial treatment of opioid overdose shifted from the emergency department to first responders (e.g., police, fire department, friends and family of overdose victims), intranasal (IN) naloxone has become the standard rescue agent in a community setting. First approved by the FDA in November 2015, IN naloxone (4 mg) can be administered with little or no training, is absorbed as rapidly as an intramuscular (IM) injection and eliminates the potential for needlestick injury. While multiple factors (such as the type and quantity of opioid, route of administration, presence of other drugs, and interval between overdose and intervention) ultimately determine a patient's prognosis, both clinical and preclinical evidence indicate that higher doses of naloxone are needed to reverse a synthetic opioid overdose than are typically used by first responders. Multiple clinical studies have reported high doses of parenteral naloxone (in some cases followed by a naloxone infusion) are required for rescue, with recommendations of up to 12-15 mg if a synthetic like fentanyl is involved. Moreover, preclinical studies examining respiratory depression in the absence of the multiple factors that complicate interpretation of a clinical overdose have demonstrated that up to ten-fold higher doses of naloxone are needed to reverse respiratory depression produced by fentanyl compared to an opium-based alkaloid like morphine despite comparable affinities at m-opioid receptors. More recent studies have provided key molecular insights which may contribute to this apparent paradox.

Opioid overdoses, particularly in the community setting, are highly heterogeneous events that depend on a variety of factors that each vary over ranges based on multiple parameters. Therefore, studies examining the effectiveness of opioid antagonists evaluating variables like doses, dosing strategies, etc. based on case studies and chart reviews are not only subject to factors which contribute to the heterogeneous nature of an overdose (e.g. ranging from type(s) and quantity(ies) of opioid(s) taken to the latency between overdose and intervention) but also to the variability in reporting outcomes (e.g. changes in respiration versus consciousness), study quality, and selection bias.

Mechanistic modeling studies present an unbiased, alternative approach to estimate the effectiveness of m-opioid receptor antagonists in a first-responder or community setting. Model-based approaches have been successfully applied in drug development and can help address information gaps when it is challenging to conduct clinical trials. A translational mechanistic model recently published by Mann et al (2022) (see, Mann J, Samieegohar M, Chaturbedi A, Zirkle J, Han X, Ahmadi S F, et al. Development of a translational model to assess the impact of opioid overdose and naloxone dosing on respiratory depression and cardiac arrest. Clin Pharmacol Ther. (2022) 112:1020-32. doi: 10.1002/cpt.2696) was developed to predict the extent of respiratory depression and incidence of cardiac arrest triggered by a synthetic opioid overdose in the absence of antagonist treatment and following administration of IM naloxone. This model was recently implemented to evaluate the efficacy of IN naloxone given as single or multiple doses.

To aid in uniform analysis and ease of interpretation of interventions to treat or reverse opioid overdose, a translational mechanistic model, of synthetic opioid overdose was developed by the FDA's Division of Applied Regulatory Science to predict the outcomes such as respiratory depression, cardiac arrest, etc. following an opioid overdose caused by potentially lethal doses of fentanyl or carfentanil. This translational mechanistic model, also referred to herein as the “Mann Model,” represents an alternative, unbiased approach to estimate the effectiveness of opioid antagonist products in reversing a synthetic opioid overdose. Initially developed to compare the effectiveness of two formulations of IM naloxone, the model was recently expanded by Strauss et al. (2024) (see, Strauss D G, Li Z, Chaturbedi A, Chakravartula S, Samieegohar M, Mann J, et al. Intranasal naloxone repeat dosing strategies and fentanyl overdose: A simulation-based randomized clinical trial was used to evaluate the effectiveness of IN naloxone dosing strategies. See, JAMA Network Open. (2024) 7:e2351839-e2351839. doi: 10.1001/jamanetworkopen.2023.51839.

### Model and Simulation Methods

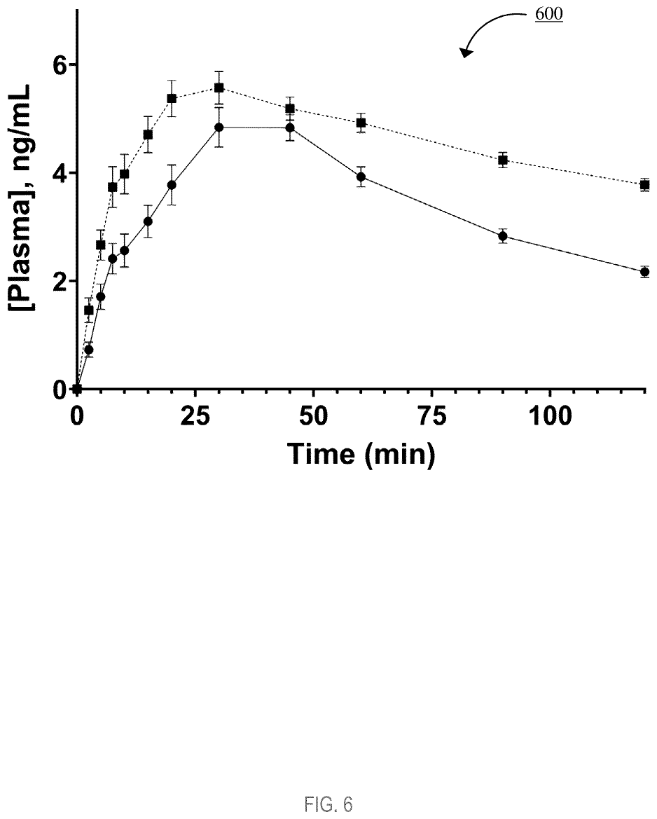

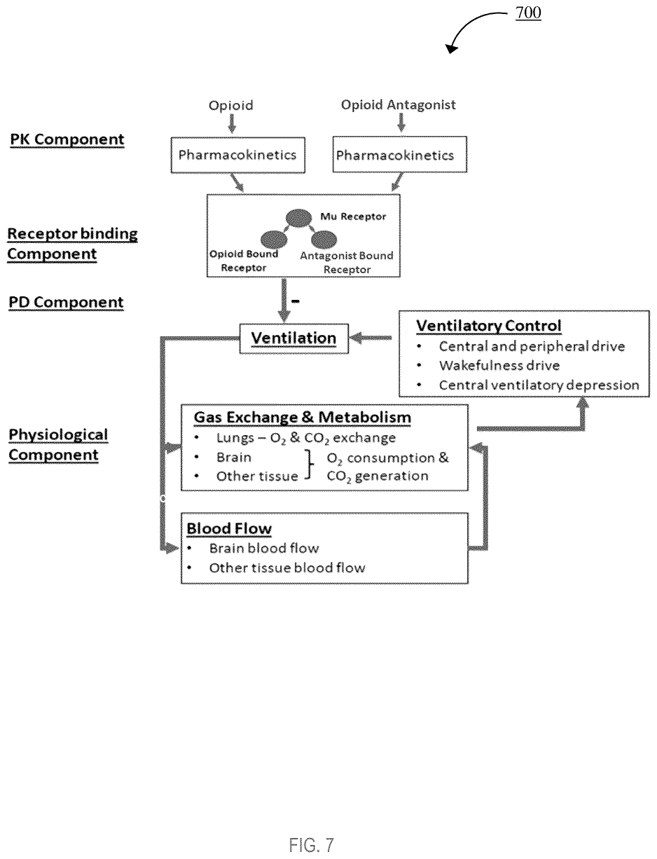

This study expanded the translational Mann model (shown schematically in FIG. 7) to include IN nalmefene and IN naloxone data. The resulting model (model 700) is also referred to herein as the “Laffont Model”. The changes to the Mann model to develop the Laffont model 700 included inclusion of pharmacokinetic data (naloxone, nalmefene) and μ-opioid receptor binding parameters (nalmefene) to simulate effects of 4 mg naloxone hydrochloride IN and 3 mg nalmefene hydrochloride IN, both FDA-approved for the treatment of opioid overdose. Model simulations were conducted to quantify the percentage of cardiac arrest in 2000 virtual patients in both the presence and absence of IN antagonist treatment.

FIG. 7 is a schematic representation of the translational mechanistic model used to compare the effectiveness of IN administration of naloxone to nalmefene, using methods disclosed herein, according to an embodiment. “Minute ventilation” of a subject sometimes referred to as “respiratory minute volume” or simply “minute volume,” is the volume of gas inhaled (called “inhaled minute volume”) or exhaled (called “exhaled minute volume”) from a subject's lungs per minute. Simulations tested the effect of IN pharmaceutical formulation including nalmefene and naloxone in treating and/or reducing adverse health effects of a synthetic opioid overdose, such as respiratory depression and cardiac arrest.

Results showed that following simulated overdoses with both fentanyl and carfentanil in chronic opioid users, using the Laffont model 700, IN nalmefene produced a substantially greater reduction in the incidence of cardiac arrest compared to IN naloxone. For example, following a dose of fentanyl (1.63 mg) producing cardiac arrest in 52.1% (95% confidence interval, 47.3-56.8) of simulated patients, IN nalmefene reduced this rate to 2.2% (1.0-3.8) compared to 19.2% (15.5-23.3) for IN naloxone. Nalmefene also produced large and clinically meaningful reductions in the incidence of cardiac arrests in opioid naïve subjects. Across dosing scenarios, simultaneous administration of four doses of IN naloxone were needed to reduce the percentage of cardiac arrest events to levels that approached those produced by a single dose of IN nalmefene. The results are explained in more detail below.

This translational model was configured among others to compare the effectiveness of IN naloxone to IN nalmefene following a synthetic (fentanyl, carfentanil) opioid overdose. The Laffont model 700 predicts adverse health effects including the extent of respiratory depression and incidence of cardiac arrest caused by a synthetic opioid (fentanyl, carfentanil) overdose in the absence of an intervention and following administration of an opioid antagonist. As illustrated in FIG. 7, the model integrates several components characterizing: (i) the pharmacokinetics of the synthetic opioid and antagonist for the route of administration investigated and their brain penetration (PK Component), (ii) the competitive binding between the synthetic opioid and antagonist at the μ-opioid receptor (Receptor binging Component), (iii) the effect of the synthetic opioid-μ-opioid receptor complex on ventilation via a reduction of ventilatory drives (central chemoreflex, peripheral chemoreflex, and wakefulness drives) (PD Component), and (iv) the physiological feedback mechanisms describing gas (oxygen [O2] and carbon dioxide [CO2]) exchange and metabolism, blood flow control, and ventilatory control (Physiological Component).

In the pharmacokinetic (PK) component, compartment PK models convert the doses of opioids and opioid antagonists administered by different routes to their free concentrations in the effective compartment. Population pharmacokinetic models (nonlinear mixed-effects model) were developed for IN nalmefene and IN naloxone using plasma concentration data from three clinical studies in healthy volunteers: (i) two pharmacokinetic studies published by Crystal et al. (see, Crystal R, Ellison M, Purdon C, Skolnick P. Pharmacokinetic properties of an FDA-approved intranasal nalmefene formulation for the treatment of opioid overdose. Clin Pharmacol Drug Dev. (2024)) assessing a single dose of 3 mg IN nalmefene HCl compared to 1 mg IM nalmefene and two doses of 3 mg IN nalmefene HCl (evaluating two doses in one nostril or one dose in each nostril), and (ii) the pharmacodynamic study described above under Study I comparing the effects of 3 mg IN nalmefene HCl and 4 mg IN naloxone HC in an experimental model of opioid-induced respiratory depression. Serial blood samples were collected to characterize the pharmacokinetics of both IN nalmefene and IN naloxone in the pharmacodynamic study. Data from the three studies were used to develop the population pharmacokinetic model for IN nalmefene. The population pharmacokinetic model for IN naloxone was developed using concentration data from the pharmacodynamic study. Detailed information on clinical Studies and methodology used for population pharmacokinetic analyses are provided in Tables 5 and 6 below.

TABLE 5
Clinical Studies Used for Population Pharmacokinetic
Modeling and Validation of the Translational Model

| Study | | | |
|---|---|---|---|
| (NLM | Study Design | Dose/Regimens | Subjects |
| PK Study 1 | Open-label, randomized, 2- | Each subject received both treatments, | 68 |
| (NCT04759768; | period, 2-treatment, 2- | a single IN dose of 3 mg nalmefene | healthy |
| Crystal et | sequence, crossover | HCl or a single IM dose of 1.0 mg | subjects |
| al., 2024) | study to determine the PK | nalmefene, by random assignment to | |
| | and safety of one dose of 3 | one of 2 possible treatment sequences; | |
| | mg nalmefene HCl IN spray | a 4-day washout period separated | |
| | compared to one dose of 1.0 | treatments. | |
| | mg nalmefene by IM | | |
| | injection. | | |
| PK Study 2 | Open-label, randomized, 6- | Subjects were randomly assigned to 1 | 24 |
| (NCT05219669; | sequence, | of 6 sequences | healthy |
| Crystal et | 3-treatment, 3-period | (4 subjects per sequence) with each | subjects |
| al., 2024 | crossover study to assess the | subject receiving all 3 regimens: | |
| | PK, safety, and tolerability | 3 mg nalmefene HCl, one 0.1 mL | |
| | of nalmefene following | spray in one nostril | |
| | administration of three IN | 6 mg nalmefene HCl, one 0.1 mL | |
| | dosing regimens | spray in each nostril | |
| | | 6 mg nalmefene HCl, two 0.1 mL | |
| | | sprays in one nostril Each drug | |
| | | administration was separated by a | |
| | | washout period of 6 days. | |
| PD Study | Open-label, randomized, 2- | Part 1 (including Part 1 extension) | Part 1: |
| (NCT04828005; | period, | 4 mg IN naloxone HCl, on Dose Day | 7 healthy |
| Ellison et | 2-treatment, crossover study | 1 and Dose Day 2, with a 4-day | subjects |
| al., 2024 | conducted in two parts (Part | washout period between doses (Study | with prior |
| | 1 and Part 2) | Day 1 and Day 5). | opioid |
| | Part 1 (including Part 1 | Part 2: | exposure |
| | extension) was a pilot study to | Each subject received both treatments, | Part 1 extension: |
| | determine the relationship | a single IN dose of 3 mg nalmefene | 8 healthy |
| | between remifentanil dose | HCl or a single IN dose of 4.0 mg | subjects |
| | and suppression of CO2- | naloxone HCl, by random assignment | with prior |
| | induced increases in minute | to one of 2 possible treatment | opioid |
| | ventilation in healthy | sequences; a 4-day washout period | exposure |
| | volunteers with prior opioid | separated treatments. | Part 2: |
| | exposure. | On each dosing day, a remifentanil IV | 69 |
| | Part 2 aimed to evaluate the | bolus dose | healthy |
| | PD effects of IN nalmefene | (0.5 μg/kg) was administered at time | subjects |
| | compared to IN naloxone in | 10 minutes, followed by a remifentanil | with prior |
| | reversing remifentanil- | infusion (rate: 0.175 μg/kg/min) which | opioid |
| | induced suppression of | continued for the study duration. | exposure |
| | CO2-induced increases in | At time 25 minutes, IN nalmefene or | |
| | minute ventilation in | IN naloxone was administered. | |
| | healthy volunteers with | | |
| | prior opioid exposure. | | |

All clinical studies were conducted in accordance with principles and requirements of the International Council for Harmonization Good Clinical Practice guidelines, Written informed consent was obtained from all participants before starting any study-related procedure. Clinical study protocols, informed consent forms, and all other appropriate study-related documents were reviewed and approved by institutional review boards.

TABLE 6
Population Pharmacokinetic Model Development for
Intranasal Nalmefene and Intranasal Naloxone

| Population | | | Covariate |
|---|---|---|---|
| PK Model | Data1 | Methodology | Analysis |
| IN and IM | 4401 | The overall procedure for population PK | A relative |
| Nalmefene | quantifiable | model development included: 1) exploratory | bioavailability |
| | nalmefene | data analysis; 2) base structural model | parameter was |
| | plasma | development; 3) evaluation of covariate | included in |
| | concentrations | effects; 4) final model refinement; and 5) | nalmefene base |
| | collected from | model evaluation. | model to |
| | 153 subjects | Results of the exploratory analyses were used | simultaneously |
| | enrolled in PK | to determine the appropriate functional form | model IN and IM |
| | studies 1 & 2 | of the base structural model of drug plasma | PK data. |
| | and the PD | concentration versus time data. | Additionally, a |
| | study (Part 2) | Due to the variability in concentration profiles | study effect on IN |
| | | during IN absorption, several alternative | absorption was |
| | | absorption models were explored, including | estimated based on |
| | | transit and sigmoid absorption, sequential | exploratory data |
| | | zero- and first-order absorption, and parallel | plots. |
| | | zero- and first- order absorption with and | A systematic |
| | | without lag times. | stepwise covariate |
| | | The same structural base model used for IN | screening examined |
| | | nalmefene was fit to IN naloxone PK data | the effects of the |
| | | collected in the PD study. As PK sampling for | following |
| | | naloxone only extended to 2 hours post dose, | stationary |
| | | there was insufficient data to estimate all | covariates based on |
| | | disposition parameters. Therefore, Q and Vp | baseline or |
| | | were fixed to population estimates from | screening |
| | | Yassen et al. (2007). | assessments: age, |
| | | Interindividual variability was estimated as | body weight, body |
| | | applicable assuming a log-normal distribution | mass index, racial |
| | | of individual PK parameters. | classification, sex, |
| | | The adequacy of the final PK models was | serum albumin, |
| | | evaluated on the basis of the plausibility and | hematocrit, alanine |
| | | precision of the parameter estimates, | aminotransferase, |
| | | goodness-of-fit plots, and using a simulation- | and aspartate |
| | | based, visual predictive check method to | aminotransferase. |
| | | assess concordance between the observed data | The forward |
| | | and model-based simulated data. | selection followed |
| | | Population PK modeling was performed using | by backward |
| | | the computer program NONMEM version | elimination |
| | | 7.3. NONMEM analyses were performed on | approach was |
| | | an Intel cluster with the Linux operating | applied to covariate |
| | | system. | model building. |
| IN | 632 | | No formal covariate |
| Naloxone | quantifiable | | analysis was |
| | naloxone | | conducted, but the |
| | plasma | | effect of body |
| | concentrations | | weight on |
| | collected from | | disposition |
| | 60 subjects | | parameters CL and |
| | enrolled in the | | Vc was assessed. |
| | PD study (Part | | |
| | 2) | | |

In the receptor binding component, the synthetic opioid and opioid antagonist compete to bind to the μ-opioid receptor. Ordinary differential equations and parameter estimates from IN nalmefene and IN naloxone pharmacokinetic models were integrated into the Mann model framework. The Mann model utilizes a competitive binding model at the μ-opioid receptor with binding parameters (association rate constant kₒₙ, dissociation rate constant k_{off}, and steepness parameter n) implemented for naloxone, fentanyl, and fentanyl derivatives including carfentanil. A scaling approach was used to estimate nalmefene binding parameters relative to naloxone based on kₒₙ and k_{off} estimates from a study by Cassel et al. (see, Cassel J A, Daubert J D, DeHaven R N. [3H] Alvimopan binding to the m-opioid receptor: comparative binding kinetics of opioid antagonists. Eur J Pharmacol. (2005) 520:29-36). The ratios of nalmefene kₒₙ to naloxone kₒₙ and of nalmefene k_{off} to naloxone k_{off} using these values were multiplied by the naloxone kₒₙ and k_{off} used in the Mann model to obtain scaled binding parameters for nalmefene, retaining the same relative potency. The steepness parameter for nalmefene, n, was assumed identical to the one for naloxone (0.86). The same plasma to brain equilibration rate constant, kₑ₀, for nalmefene and naloxone were also adopted given similar physicochemical properties, using the value of 0.001774 sec−1 from the Mann model. Table 7 below summarizes pharmacokinetic and binding parameters for naloxone and nalmefene submodels.

TABLE 7
Pharmacokinetic and Pharmacodynamic Parameters of Nalmefene,
Naloxone and Remifentanil Used in the Translational Model

| | | Estimate | |
|---|---|---|---|
| Compound | Parameter(s) | (Variability CV %) | Source |
| IN | CL/F, Vc/F, Q/F, | See Table 9 | Clinical data (see Table 6) |
| nalmefene | Vp/F, INKA, D2, | | |
| | INFK0, ALAG1, | | |
| | FR, s2 | | |
| | kₑ₀ (sec⁻¹) | 0.001774 | Assumed identical to ke0 for |
| | | | naloxone in Mann et al. (2022) |
| | kₒₙ (pM-nsec⁻¹) | 2.06E−04 | Cassel et al. (2005) and Mann et al. |
| | | | (2022)1 |
| | k_{off} (sec⁻¹) | 1.35E−02 | Cassel et al. (2005) and Mann et al. |
| | | | (2022)2 |
| | n | 0.86 | Assumed identical to naloxone |
| | | | (Mann et al., 2022) |
| IN naloxone | CL/F, Vc/F, Q/F, | See Table 2 | Clinical data (see Table 6) |
| | Vp/F, KA, D2, | | |
| | FK0, ALAG1, | | |
| | s2 | | |
| | kₑ₀ (sec⁻¹) | 0.001774 | Mann et al. (2022) |
| | kₒₙ (pM-nsec⁻¹) | 1.67E−04 | |
| | k_{off} (sec⁻¹) | 3.96E−02 | |
| | n | 0.86 | |
| IV | CL (L/min) | 2.58 (14.1%) | Eleveld et al. (2017) |
| remifentanil | Q1 (L/min) | 1.72 (23.7%) | |
| | Q2 (L/min) | 0.124 (57.5%) | |
| | V1 (L) | 5.81 (33.0%) | |
| | V2 (L) | 8.82 (35.0%) | |
| | V3 (L) | 5.03 (112%) | |
| | kₑ₀ (sec⁻¹) | 0.0218 | Olofsen et al. (2010)3 |
| | kₒₙ (pM-nsec⁻¹) | 8.08E−06 | Mann et al. (2022) |
| | k_{off} (sec⁻¹) | 2.08E−03 | |
| | n | 0.70 (changed to | |
| | | 0.75 in the final | |
| | | model) | |

### Model Validation

The original Mann model was developed and validated using an extensive set of data, including in-vitro μ-opioid receptor binding data, animal studies, and clinical studies assessing the respiratory effects of synthetic opioids in chronic opioid users and opioid-naïve healthy volunteers.

Model simulations were conducted to assess if the expanded Laffont model 700 acceptably reproduced minute ventilation recovery data in the pharmacodynamic study described above in Study I, using the clinical model of opioid-induced respiratory depression in hypercapnia-induced raise in minute ventilation. Briefly, as described in Study 1, subjects breathed a hypercapnic gas mixture (50% oxygen, 43% nitrogen, 7% carbon dioxide) to elevate minute ventilation. Ten minutes after initiating the hypercapnic gas mixture, remifentanil was administered as an IV bolus dose of 0.5 mg/kg followed by an IV infusion at the rate of 0.175 mg/kg/min which was continued for the duration of the study. Fifteen minutes after initiating remifentanil infusion (defined as the nadir of minute ventilation), subjects received either 3 mg IN nalmefene HCl or 4 mg naloxone HC and were monitored for another 21 minutes.

Simulations were performed in virtual opioid naïve users since Study I was conducted with opioid experienced but nondependent healthy volunteers. A single typical subject with mean parameter values generating an “average” profile was simulated for comparison to average measures of ventilation observed in the study. A remifentanil population pharmacokinetic model published by Eleveld et al. (see, Eleveld D J, Proost J H, Vereecke H, Absalom A R, Olofsen E, Vuyk J, et al. An allometric model of remifentanil pharmacokinetics and pharmacodynamics. Anesthesiology. (2017) 126:1005-18.doi: 10.1097/ALN.0000000000001634) and remifentanil binding parameters from the Mann model (kₒₙ, 8.08E-06 pM-nsec−1; k_{off}, 2.08E-03 sec−1; n, 0.70) were used for simulations.

The plasma to brain equilibration rate constant for remifentanil (kₑ₀) was fixed to 0.0218 sec−1 based on the equilibration half-life of 0.53 minutes reported by Olofsen et al. (see, Olofsen E, Boom M, Nieuwenhuijs D, Sarton E, Teppema L, Aarts L, et al. Modeling the non-steady state respiratory effects of remifentanil in awake and propofol-sedated healthy volunteers. Anesthesiology. (2010) 112:1382-95. doi: 10.1097/ALN.0b013e3181d69087). Remifentanil parameters are summarized in Table 7 above.

Modifications of the code were performed to induce a hypercapnic state similar to that observed in the pharmacodynamic study. Specifically, the CO₂ partial pressure was altered by changing the “P_a_co2” (arterial CO₂ partial pressure) parameter from 40.28 to 45.30 millimeters of mercury (mm Hg) (using script “delaystates.R”). In addition, to allow the partial pressure of CO2 to vary during the simulation, the parameters “P_i_co2” (inspired CO2) and “P_a_co2” were changed from 0 to 34 mm Hg and 40 to 45.30 mm Hg, respectively (delaypars.R script).

Opioid overdose simulations were conducted with the final Laffont model 700 to compare the incidence of cardiac arrest following rescue treatment with IN nalmefene compared to IN or IM naloxone. While the model was configured to simulate multiple physiological outcomes resulting from opioid-induced respiratory depression, Mann et al. (2022) focused on cardiac arrest as an endpoint because in a community setting, the cardiovascular complications produced by asphyxia (the hypoxia and hypercapnia resulting from respiratory depression) are inevitably fatal in the absence of intervention. For context, the incidence of opioid-associated out-of-hospital cardiac arrests (OHCA) has been estimated at between 6-14% of all OHCA treated by emergency services personnel. However, these estimates generally include only cases that received cardiopulmonary resuscitation, excluding both decedents with a presumed opioid overdose who did not receive cardiopulmonary resuscitation and overdose fatalities where emergency services were not provided.

In the presented study II, multiple variables were explored in the simulations, such as the opioid responsible for the overdose (fentanyl or carfentanil), the opioid dose, antagonist dose, and the type of opioid user (chronic vs. naïve). The percentage of subjects with opioid-induced cardiac arrest from a population of 2000 virtual subjects was used as the outcome of interest for each opioid receptor antagonist formulation and dosing regimen. As in the Mann model, cardiac arrest was defined as occurring when the total blood flow reached a value of 0.01 L/min.

For fentanyl and carfentanil, the same IV bolus doses as in the Mann model were simulated to represent “medium” and “high” overdose severities (1.63 mg and 2.97 mg for fentanyl and 0.012 mg and 0.022 mg for carfentanil, respectively). In order to provide additional context for the doses of fentanyl used in these simulations: a 2 mg dose of fentanyl is considered a potentially lethal dose by the DEA, with 70% of counterfeit pills seized in 2023 containing a lethal dose. The DEA has reported a range of 0.02-5.1 mg of fentanyl per counterfeit tablet; an independent report using a very small sample of counterfeit pills reported between 0.6-6.9 mg of fentanyl in a single ‘batch’, confirming the high content variability of illicitly manufactured synthetic opioids.

As in the Mann model, a residual minute ventilation volume of 40% of baseline was used as the threshold of respiratory depression to trigger administration of the opioid antagonist (naloxone or nalmefene) which occurred with a 1-minute delay to account for potential time lost for product preparation.

Different dosing scenarios for the opioid antagonist were evaluated, including (i) administration of 1 or 2 doses of IN nalmefene (corresponding to 3 mg and 6 mg of the HCl salt, respectively), (ii) administration of 1, 2, 3, or 4 doses of IN naloxone (corresponding to 4 mg, 8 mg, 12 mg and 16 mg of the 1-CI salt, respectively), and (iii) administration of IM naloxone using the commercially available 2 mg/2 mL formulation. When multiple doses were simulated for IN naloxone or IN nalmefene, doses were administered simultaneously.

The Mann model describes the application of bootstrapping methods for generating a distribution of kₒₙ, k_{off}, and n estimates for naloxone, fentanyl, and carfentanil. The variability in experimental data and parameter uncertainty in binding parameters were accounted for by using the distributions of binding parameters (N=2000) generated by Mann et al. (2022) and provided in an associated GitHub public repository. Nalmefene binding parameters were generated based on the distribution of naloxone binding parameters using the scaling approach described above. The 2000 sets of pharmacokinetic parameters simulated for nalmefene and naloxone were combined with the 2000 sets of pharmacodynamic binding parameters to generate 2000 virtual subjects.

A bootstrap resampling method was used to estimate the summary statistics (median, and 2.5^{th} and 97.5^{th} percentiles defining a 95% confidence interval) for the incidence of cardiac arrest. A sample with size of 400 was drawn 2500 times from the 2000 virtual subjects simulated for each dosing scenario. The cardiac arrest rate was estimated for each of the 2500 bootstrap samples.

Results showed that plasma concentrations following IN administration of nalmefene and naloxone were best fitted to 2-compartment models with linear elimination and parallel zero- and first-order absorption with a lag time at the start of the first-order absorption process. The adequacy of the pharmacokinetic models was demonstrated by visual predictive checks showing good concordance between observed plasma concentrations and model predictions.

Model parameter estimates are displayed in Table 8 for IN nalmefene and in Table 9 for IN naloxone, below.

TABLE 8
Final Population Pharmacokinetic Model for
Intranasal (and Intramuscular) Nalmefene

| | | Final Param. | | Magnitude of Variability | |
|---|---|---|---|---|---|
| | | Population | | Final | |
| Parameter | | Mean | % RSE | Estimate | % RSE |
| CL/F | Apparent clearance | 63.7 | 2.1 | 15.4% CV | 19.3 |
| | (L/h) | | | | |
| | Exponent of (WT/74.7) | 0.572 | 16.6 | — | — |
| | for CL/F | | | | |
| Vc/F | Apparent volume of | 15.2 | 11.8 | 211% CV | 10.3 |
| | distribution, central | | | | |
| | compartment (L) | | | | |
| Q/F | Apparent clearance of | 81.3 | 7.23 | — | — |
| | distribution (L/h) | | | | |
| Vp/F | Apparent volume of | 522 | 3.05 | — | — |
| | distribution, peripheral | | | | |
| | compartment (L) | | | | |
| INKA | Intranasal first-order | 0.497 | 9.09 | 39.8% CV | 18.4 |
| | absorption rate | | | | |
| | constant (1/h) | | | | |
| IMKA | Intramuscular first- | 0.156 | 5.45 | 50.4% CV | 18.4 |
| | order absorption rate | | | | |
| | constant (1/h) | | | | |
| D2 | Zero-order absorption | 0.302 | 7.33 | — | — |
| | duration (h) | | | | |
| INFK0 | Fraction of intranasal | 0.0485 | 13.3 | — | — |
| | dose with zero-order | | | | |
| | absorption | | | | |
| IMFK0 | Fraction of | 0.017 | 10.9 | — | — |
| | intramuscular dose | | | | |
| | with zero-order | | | | |
| | absorption | | | | |
| ALAG1 | Lag-time of first-order | 0.0615 | 7.58 | — | — |
| | absorption (h) | | | | |
| FR | Relative bioavailability | 0.834 | 2.23 | — | — |
| | for intranasal vs. | | | | |
| | intramuscular route | | | | |
| STDEFF | Proportional shift in | −0.349 | 17.7 | — | — |
| | INKA in the | | | | |
| | pharmacodynamic | | | | |
| | study | | | | |
| s2 | Residual variability | 0.111 | 4.48 | 33.3% CV | — |

Body weight was identified as a statistically significant covariate on nalmefene apparent clearance (CL/F) with no clinical relevance. Additionally, the model predicted a slower absorption of IN nalmefene in the pharmacodynamic study, with a 35% decrease in first-order absorption rate compared to the two other pharmacokinetic studies also conducted in healthy volunteers. This decrease was attributed to the drying effect of breathing a hypercapnic mixture on the nasal mucosa, which blunted the effects of the nasal absorption enhancer, dodecyl maltoside. To reflect the use of IN nalmefene in a rescue setting, absorption parameters estimated in the absence of a mask delivering a hypercapnic gas mixture were used for opioid overdose simulations. The experimental conditions in the pharmacodynamic study did not affect IN absorption of naloxone during the first critical 20 minutes post dose when comparing these data to published data in healthy volunteers.

TABLE 9
Final Population Pharmacokinetic Model for Intranasal Naloxone

| | | Final Param. | | Magnitude of Variability | |
|---|---|---|---|---|---|
| | | Population | | Final | |
| Parameter | | Mean | % RSE | Estimate | % RSE |
| CL/F | Apparent clearance | 396 | 5.50 | 39.1% CV | 22.4 |
| | (L/h) | | | | |
| | Exponent of | | | | |
| | (WT/74.7) for CL/F | | | | |
| Vc/F | Apparent volume of | 65.7 | 24.0 | 240% CV | 22.6 |
| | distribution, central | | | | |
| | compartment (L) | | | | |
| Q/F | Apparent clearance of | 284 (fixed)¹ | — | — | — |
| | distribution (L/h) | | | | |
| Vp/F | Apparent volume of | 102 (fixed)¹ | — | — | — |
| | distribution, | | | | |
| | peripheral | | | | |
| INKA | Intranasal first-order | 0.998 | 10.6 | — | — |
| | absorption rate | | | | |
| | constant (1/h) | | | | |
| D2 | Zero-order | 0.689 | 3.67 | — | — |
| | absorption duration | | | | |
| | (h) | | | | |
| INFK0 | Fraction of intranasal | 0.183 | 23.1 | 151% CV | 30.1 |
| | dose with zero-order | | | | |
| | absorption | | | | |
| ALAG1 | Lag-time of first- | 0.0717 | 1.82 | — | — |
| | order absorption (h) | | | | |
| STDEFF | Residual variability | 0.104 12.2 | 0.104 12.2 | 0.104 12.2 | 0.104 12.2 |
| s2 | | 32.3% CV - | 32.3% CV | 32.3% CV - | 32.3% CV - |

Values in Tables 8 and 9 show % CV, coefficient of variation expressed as a percentage. % RSE indicates relative standard error expressed as a percentage. Marker ¹ indicates population means of Q/F and Vp/F were fixed to values estimated by Yassen et al. (see Yassen A, Olofsen E, van Dorp E, Sarton E, Teppema L, Danhof M, et al. Mechanism-based pharmacokinetic-pharmacodynamic modelling of the reversal of buprenorphine-induced respiratory depression by naloxone: a study in healthy volunteers. Clin. Pharm. (2007) 46:965-80).

Once the translational model was expanded with parameters for IN nalmefene and IN naloxone, the predictive validity of the model was assessed by simulation of the pharmacodynamic study (Study I) which compared the efficacy of IN nalmefene and IN naloxone in reversing remifentanil-induced respiratory depression. Here, simulations accounted for the slower absorption of IN nalmefene resulting from experimental study conditions as described above.

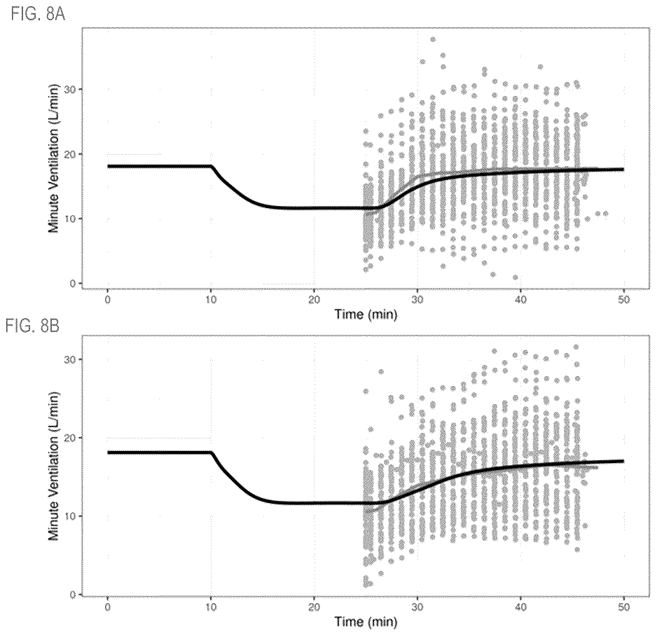

Plots in FIGS. 8A and 8B show the reproducibility of minute ventilation data in the pharmacodynamic study I described above, assessing the effect of intranasal nalmefene (FIG. 8A) and intranasal naloxone (FIG. 8B), as a function of time, in simulations using the Laffont model 700, using methods and/or pharmaceutical formulations disclosed herein, according to an example implementation. The clinical data represented by the scatter plot show the effect of IN opioid antagonists in reversing respiratory depression induced by remifentanil. At 10 minutes, subjects were administered remifentanil as an intravenous bolus (0.5 mg/kg) followed by an infusion (rate 0.175 mg/kg/min) continuing until the end of the study. At 25 minutes time, subjects received either 3 mg IN nalmefene HCl (FIG. 8A) or 4 mug IN naloxone HCl (FIG. 8B) to reverse remifentanil-induced respiratory depression. As described above under study II, the study was conducted under hypercapnic conditions, with subjects breathing a hypercapnic gas mixture (50% oxygen, 43% nitrogen, 7% carbon dioxide) through a tight-fitting mask. The black curve represents the model predictions for a typical virtual opioid naïve individual, the red line is the average curve based on observed clinical data, and the closed grey circles are the observed clinical values.

As shown in FIGS. 8A and 8B, the model closely reproduced the change in minute ventilation in the clinical study with minor adjustment to the steepness parameter for remifentanil (changed from 0.70 to 0.75). No adjustments were made to either nalmefene or naloxone pharmacokinetic or binding parameters, demonstrating the robustness of the model in describing respiratory outcomes for both opioid antagonists.

The Laffont model 700 was then applied to compare cardiac arrest outcomes following administration of IN nalmefene and IN or IM naloxone. In virtual chronic opioid users, IN nalmefene resulted in a substantially greater reduction in the incidence of cardiac arrest (Table 11). For example, following an IV fentanyl dose of 1.63 mg resulting in cardiac arrest in 52.1% (95% confidence interval: 47.3-56.8) of simulated patients, IN nalmefene 3 mg reduced this rate to 2.2% (1.0-3.8) compared to 19.2% (15.5-23.3) for IN naloxone 4 mg and 29.5% (25.3-34.0) for IM naloxone 2 mg/2 mL. At a higher IV fentanyl dose (2.97 mg) producing cardiac arrest in 77.9% (73.8-81.8) of simulated patients, IN nalmefene 3 mg reduced this rate to 11.6% (8.5-14.8) compared to 47.1% (42.0-52.3) for IN naloxone 4 mg and 54.2% (49.5-59.0) for IM naloxone 2 mg/2 mL. Simultaneous administration of four doses of IN naloxone (4×4 mg) was needed to reduce the incidence of cardiac arrest to values approaching those obtained with a single dose of IN nalmefene, with 3.8% (2.0-5.8) after 1.63 mg fentanyl and 17.0% (13.5-20.8) after 2.97 mg fentanyl (Table 11). Two simultaneous doses of IN nalmefene (2×3 mg) further reduced the simulated cardiac arrest percentage to 0.35% (0-1.0) and 3.8% (2.0-5.5) following fentanyl doses of 1.63 mg and 2.97 mg, respectively.

TABLE 11
Simulated Incidence of Cardiac Arrest by Opioid,
Opioid Dose, Antagonist, Antagonist Dose, and
Route of Administration in Chronic Opioid Users

| | Fentanyl | Fentanyl | Carfentanil | Carfentanil |
|---|---|---|---|---|
| | 1.63 mg | 2.97 mg | 0.012 mg | 0.022 mg |
| No | 52.1% (47.3- | 77.9% (73.8- | 59.2% (54.0- | 90.2% (87.3- |
| antagonist | 56.8) | 81.8) | 63.8) | 93.0) |

Intramuscular Simulations

| | | | | |
|---|---|---|---|---|
| 2 mg/2 mL | 29.5% (25.3- | 54.2% (49.5- | 36.6% (32.0- | 73.7% (69.5- |
| naloxone1 | 34.0)2 | 59.0) | 41.3) | 77.8) |

Intranasal Simulations

| | | | | |
|---|---|---|---|---|
| 4 mg | 19.2% (15.5- | 47.1% (42.0- | 27.5% (23.0- | 70.6% (65.8- |
| naloxone | 23.3) | 52.3) | 31.8) | 75.0) |
| 2 × 4 mg | 10.5% (7.5- | 31.8% (27.3- | 15.8% (12.3- | 57.0% (52.0- |
| naloxone | 13.5) | 36.5) | 19.4) | 61.8) |
| 3 × 4 mg | 6.6% (4.3- | 22.6% (18.4- | 10.4% (7.5- | 46.6% (42.0- |
| naloxone | 9.3) | 26.8) | 13.3) | 51.5) |
| 4 × 4 mg | 3.8% (2.0- | 17.0% (13.5- | 6.8% (4.5- | 38.9% (34.0- |
| naloxone | 5.8) | 20.8) | 9.3) | 43.5) |
| 3 mg | 2.2% (1.0- | 11.6% (8.5- | 3.8% (2.0- | 32.0% (27.5- |
| nalmefene | 3.8) | 14.8) | 5.8) | 36.8) |
| 2 × 3 mg | 0.35% (0- | 3.8% (2.0- | 0.70% (0- | 14.5% (11.0- |
| nalmefene | 1.0) | 5.5) | 1.6) | 18.3) |

In opioid naïve individuals with no pre-existing tolerance to the pharmacological actions of opioids, simulations revealed a higher percentage of cardiac arrest compared to chronic opioid users (Table 12). Like the simulation outcomes in chronic opioid users, IN nalmefene administration resulted in marked reductions in the incidence of cardiac arrest compared to IN naloxone across all dosing scenarios. For example, at a fentanyl dose of 1.63 mg which resulted in cardiac arrest in 74.7% (70.3-78.8) of simulated subjects, IN nalmefene reduced this rate to 7.6% (5.0-10.3) compared to 39.1 (34.3-43.8) for IN naloxone and 48.3% (43.5-53.3) for IM naloxone.

TABLE 12
Simulated Incidence of Cardiac Arrest by Opioid, Opioid
Dose, Antagonist, Antagonist Dose, and Route of Administration
in Opioid Naïve Individuals

| | Fentanyl | Fentanyl | Carfentanil | Carfentanil |
|---|---|---|---|---|
| | 1.63 mg | 2.97 mg | 0.012 mg | 0.022 mg |
| No antagonist | 74.7% (70.3- | 90.1% (87.0- | 75.9% (71.5- | 96.4% (94.5- |
| | 78.8) | 93.0) | 80.0) | 98.0) |

Intramuscular Simulations

| | | | | |
|---|---|---|---|---|
| 2 mg/2 mL | 48.3% (43.5- | 71.8% (67.3- | 56.6% (51.8- | 86.4% (83.0- |
| naloxone1 | 53.3) | 76.0) | 61.3) | 89.8) |

Intranasal Simulations

| | | | | |
|---|---|---|---|---|
| 4 mg IN | 39.1% (34.3- | 67.6% (63.0- | 50.5% (45.8- | 85.8% (82.3- |
| naloxone | 43.8) | 72.0) | 55.5) | 89.0) |
| 2 × 4 mg IN | 23.8% (19.6- | 53.0% (48.3- | 37.1% (32.3- | 77.1% (73.0- |
| naloxone | 27.8) | 57.5) | 41.8) | 81.3) |
| 3 × 4 mg IN | 16.3% (12.5- | 42.4% (37.8- | 26.8% (22.5- | 69.8% (65.3- |
| naloxone | 19.8) | 47.3) | 31.3) | 74.0) |
| 4 × 4 mg IN | 11.8% (8.8- | 33.4% (28.8- | 20.6% (16.5- | 63.5% (58.8- |
| naloxone | 14.8) | 37.8) | 24.8) | 68.0) |
| 3 mg IN | 7.6% (5.0- | 25.7% (21.5- | 14.9% (11.5- | 55.0% (50.0- |
| nalmefene | 10.3) | 30.0) | 18.6) | 60.0) |
| 2 × 3 mg IN | 1.7% (0.5- | 10.1% (7.3- | 5.8% (3.8- | 36.8% (32.1- |
| nalmefene | 3.0) | 13.0) | 8.3) | 41.8) |

Like the results observed in case of chronic opioid users, simultaneous administration of four doses of IN naloxone was needed in opioid naïve individuals to reduce the incidence of cardiac arrest to values approaching those obtained with one dose of IN nalmefene (Table 12). Qualitatively similar outcomes were obtained following IV carfentanil in both chronic opioid users and opioid naïve individuals, although the percentage of simulated cardiac arrests was uniformly higher for carfentanil than for fentanyl because of its much higher affinity for and slower dissociation from m-opioid receptors (Tables 11 and 12).

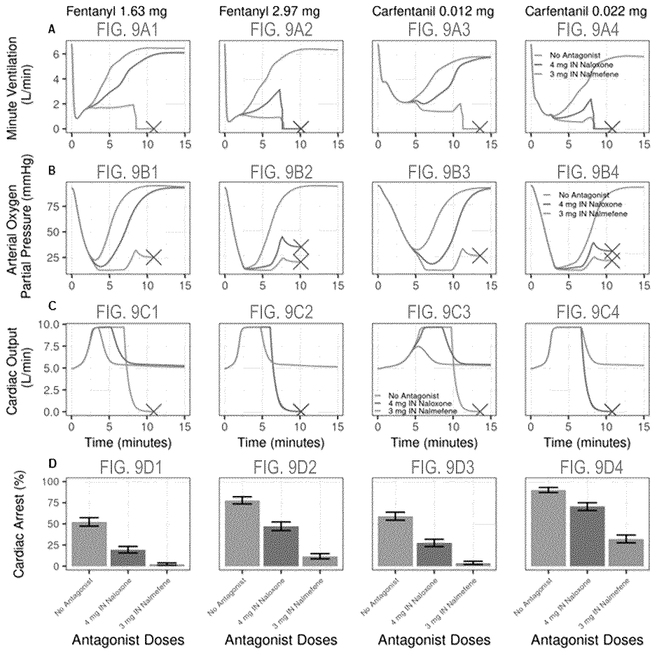

FIGS. 9A1-9A4, 9B1-9B4, and 9C1-9C4 illustrate results obtained from model simulations evaluating the effect of intranasal nalmefene versus intranasal naloxone on physiological variables and cardiac arrest following an intravenous bolus dose of fentanyl or carfentanil in chronic opioid users. Simulated minute ventilation (A), arterial oxygen partial pressure (B), and cardiac output (C) are plotted as a function of time in minutes, in FIGS. 9A1-9A4, 9B1-9B4, and 9C1-9C4, respectively, for a typical virtual subject who is a representative chronic opioid user. The red X designates when a typical virtual subject had a complete cardiac arrest (that is, total blood flow near zero), which terminates the simulation. FIGS. 9D1-9D4 in row (D) show the simulated percentage of virtual subjects experiencing cardiac arrest. A single dose of IN naloxone 4 mg (green) is compared with a single dose IN nalmefene 3 mg (blue) and no antagonist (red). Error bars represent the 2.5th and 97.5th percentiles after randomly sampling 400 out of the 2000 virtual chronic opioid users 2500 times.

### Conclusion

A growing body of data both preclinical and clinical indicates that higher doses of naloxone are needed to reverse a synthetic opioid overdose. Consistently, State Unintentional Drug Overdose Reporting System (SUDORS) database indicates that some form of naloxone had been administered in more than 20% of opioid-related overdose deaths in 2022; in some jurisdictions, more than 35% of decedents had received naloxone. Nonetheless, there is resistance to using either higher doses of naloxone or more potent and rapid onset agents like nalmefene. Much of this resistance may be driven by the potential for more severe withdrawal in patients with a pre-existing opioid use disorder based on reports that “standard doses of naloxone” needed to reverse an overdose have not risen significantly despite the demonstrable rise in synthetic opioids in the illicit drug supply. However, conclusions based on anecdotal observations, case studies, and chart reviews suffer from the inherent heterogeneity of an opioid overdose, variability in reporting outcomes, and study quality. For example, multiple reports used to support the position that naloxone doses have not increased attempted to standardize IN, IV, and IM naloxone dosing as “naloxone IV equivalents” based on bioavailability. While the attempt to standardize naloxone dosing based on plasma exposure is understandable, it does not account for differences in early plasma concentrations across routes of administration, which is essential for the successful reversal of a synthetic opioid overdose. These reports may also introduce a significant reporting bias, with reported EMS rescue rates of ≥99% which underestimate opioid overdose deaths reported in the SUDORS database. The use of a validated translational model of opioid overdose offers an unbiased approach to estimating the effectiveness of naloxone in the face of a potentially lethal opioid overdose.

The Laffont model 700 evaluated the effectiveness of IM naloxone in reversing hypoxia-induced cardiac arrest produced by fentanyl and carfentanil, and showed that the then standard IM doses of naloxone (2 mg/2 mL) favored by many first responders may result in a significant loss of life following an IV synthetic opioid overdose. Fentanyl doses of 1.63 mg and 2.97 mg were modeled because these doses result in plasma concentrations similar to the mean and one standard above the mean plasma concentrations, respectively, measured post-mortem in a study of approximately 500 fatal fentanyl overdoses. In the Mann model, an IV bolus of 1.63 mg fentanyl produced cardiac arrest in approximately 52% of 2000 virtual chronic opioid users in the absence of antagonist treatment. Following IM administration of naloxone (2 mg/2 mL.), the incidence of cardiac arrest was reduced to 30%, corresponding to a rescue rate of 42%. The Laffont model predicted that IM naloxone will be even less effective at reducing the incidence of cardiac arrest following administration of either 2.97 mg fentanyl (rescue rate of 30%) or the ultrapotent synthetic opioid, carfentanil (rescue rates of 18% to 38% depending on the carfentanil dose: 0.022 mg or 0.012 mg, respectively) (Table 11).

Because of the widespread use of IN naloxone in a community setting, this study implemented the expanded Laffont model 700 to compare the effectiveness of IN nalmefene to standard doses of IN naloxone HCl (4 mg), the latter generally considered the “gold standard” for first responders. The Laffont model 700 predicted large and clinically significant reductions in the incidence of cardiac arrest following rescue with IN nalmefene compared to IN naloxone under each of four conditions (i.e., medium and high doses of fentanyl and carfentanil) and for each population investigated (chronic opioid users and opioid naïve individuals) (Tables 11, 12). Following a simulated IV overdose with 1.63 mg fentanyl producing cardiac arrest in approximately 52% of patients in the absence of antagonist treatment, the incidence of cardiac arrest following IN nalmefene was 2.2% (rescue rate of >95%) compared to 19.2% following IN naloxone (rescue rate of 63%). Large differences in the effectiveness of IN nalmefene compared to IN naloxone were also evident in overdoses simulating a higher fentanyl dose as well as both the medium and high doses of carfentanil.

The “medium” and “high” doses of fentanyl used in the simulations were based on data from approximately 500 cases of fatal fentanyl overdoses, the great majority with a history of opioid use and/or chronic pain. Tolerance to the respiratory effects of opioids is known to develop following repeated use, and the incidence of cardiac arrest in this population would be predicted to be lower than those in an opioid naïve population. Implementing the model in opioid naïve individuals confirmed this prediction, with a higher incidence of cardiac arrest following both fentanyl and carfentanil administration compared to simulations in an opioid-tolerant population. Nonetheless, the differences in effectiveness between nalmefene and naloxone were as robust in this population as in simulated chronic opioid users. This is notable because the increased availability of synthetic opioids in counterfeit pills has fundamentally changed the demographics of overdose, with increased numbers of opioid naïve individuals, especially adolescents, exposed to potentially lethal doses of synthetic opioids.

Because the model simulations demonstrated large reductions in the incidence of cardiac arrest after a single dose of IN nalmefene compared to a single dose of TN naloxone, this study also examined the rates of cardiac arrest following administration of multiple doses of naloxone. Simultaneous dosing of two, three and four doses of naloxone (total amount of 8, 12, and 16 mg) produced dose-related reductions in the incidence of cardiac arrest. However, at both medium and high doses of fentanyl or carfentanil, four doses of TN naloxone (16 mg) administered simultaneously were needed to reduce the incidence of cardiac arrest to values approaching a single dose of nalmefene (Tables 11, 12).

In the Mann model, μ-opioid receptor antagonists were administered one minute after ventilation was decreased to 40% of baseline to mimic a delay between recognizing respiratory depression and administering the reversal agent. In practice, recognizing the signs of an opioid overdose requires training, and there is very often a longer delay in administering the reversal agent. Therefore, with the assumption that an opioid overdose would be immediately recognized and treated, the Laffont model predictions likely overestimate the effectiveness of both m-opioid receptor antagonists in a field setting by not accounting for potential delay in intervention. Also, while fentanyl remains the predominant synthetic found in DEA seizures, the illicit drug supply varies over time and by region. Thus, the quantities of fentanyl used in the Mann model may not reflect either the actual quantities or composition of opioids (including other opioids such as heroin) in a real-world scenario.

Overall, the difference in the apparent effectiveness between IN nalmefene and IN naloxone is consistent with preclinical and clinical evidence including: a higher affinity of nalmefene at m-opioid receptors, higher plasma concentrations of nalmefene delivered at the critical early time points (e.g., 5 min) after dosing, and a more rapid onset of action as demonstrated in a clinical model of opioid-induced respiratory depression.

Nalmefene has a significantly longer plasma half-life (t½ of 7.1-11 h) than naloxone (t½~2 h) as well as higher affinity at in-opioid receptors which have the potential risk to produce a more severe and longer precipitated withdrawal in individuals with an opioid use disorder. A double-blind, randomized clinical study comparing IV doses of naloxone (2 mg) and nalmefene (1 or 2 mg) in 176 patients admitted to nine emergency departments with suspected narcotic overdose reported that both opioid antagonists produced rapid and robust reversals of respiratory depression in patients with a confirmed opioid overdose. Adverse events were noted in opioid-positive patients in all three treatment arms, but the overall difference among treatment arms was not significant (p>0.27) and no significant overall time-treatment interactions (measured out to 240 min post-dosing) emerged; no statistical differences in withdrawal outcomes were seen between treatment groups. It is noteworthy that the overall incidence of adverse events following IV administration of these opioid antagonists is lower than in a contemporary study using information provided by first responders and community-based organizations following rescue with 4 mg IN naloxone. Furthermore, the higher plasma naloxone concentrations which may be needed to reverse a synthetic opioid overdose also carries an increased risk of precipitated withdrawal. Multiple doses of IN naloxone administered using a standard regimen (i.e., a dose administered every 2-5 minutes if there is no response) however, increases the risk of sustained precipitated withdrawal without conferring significant therapeutic advantage compared to a single IN dose.

Whether the risks of underdosing with an opioid antagonist outweigh the risks associated with precipitated withdrawal remains a matter of debate. Although the majority of opioid overdoses are not fatal, every overdose is potentially lethal in the absence of intervention and the primary goal of opioid antagonist treatment is to prevent death. While withdrawal symptoms precipitated by an opioid antagonist can be unpleasant and distressing, they are medically manageable and rarely life threatening. As such, they should not limit the use of more effective reversal strategies, especially when the odds of a synthetic opioid overdose are high On the other hand, there are concerns that precipitated withdrawal could pose acute risks to the patient and medical personnel and could trigger further opioid use to counter the effects of the opioid antagonist. Additionally, the knowledge and avoidance of precipitated withdrawal could reduce both use and acceptance of the reversal agent among patients with an opioid use disorder. Nonetheless, recent findings (see, Strickland J C, Marks K R, Smith K E, Ellis J D, Hobelmann J G, Huhn A S. Patient perceptions of higher-dose naloxone nasal spray for opioid overdose. Int J Drug Policy. (2022) 106:103751. doi: 10.1016/j.drugpo.2022.103751) indicate that while the concerns about precipitated withdrawal are valid, the majority of people who have had a lived experience accept the need for effective reversal agents for themselves and others within a community setting.

In conclusion, simulations using a validated translational model of opioid overdose demonstrate that a single dose of IN nalmefene produces clinically meaningful reductions in the incidence of cardiac arrest compared to naloxone tools (IN, IM) frequently used by first-responders across a variety of scenarios involving fentanyl and carfentanil. These findings are consistent with converging lines of pharmacological evidence that the rapid delivery of high concentrations of a potent reversal agent favors a successful rescue in the era of synthetic opioids.

Study III: Using a Validated Translational Model to IN Pharmaceutical Formulations to Treat Synthetic Opioid Overdose at Lower Dosing and/or Delayed Intervention Scenarios

The Laffont Model 700 simulates administration of IM naloxone 1-minute after a victim's minute ventilation drops to 40% of a baseline value associated with the subject, a threshold considered an “unsafe” level of opioid-induced respiratory depression. This 1-minute delay in naloxone administration was based on the time needed to prepare and deliver an IM administration following bystander recognition of an overdose. However, both the speed at which a potentially lethal dose of a synthetic opioid depresses respiration and the potential for a longer delay in recognizing the signs of an overdose prompted additional studies and model extension, and using the Laffont model 700 to examine: (i) how delays in the interval between detection of an opioid overdose and rescue changes the effectiveness of IN nalmefene and IN naloxone, (ii) the impact of fentanyl and carfentanil doses on cardiac arrest outcomes, and (iii) the impact of intervention on the duration of brain hypoxia following either a fentanyl or carfentanil overdose.

Outlined herein are example studies conducted through simulations to explore the effectiveness of IN naloxone compared to IN nalmefene in three domains: (i) following an overdose including a variable quantity of synthetic opioid; (ii) varying the time between overdose and rescue; and (iii) reducing the duration of brain hypoxia following a synthetic opioid overdose.

In the example studies, the Laffont Model 700 was used to conduct a series of simulations investigating delays in intervention with IN nalmefene or IN naloxone following detection of an overdose with either fentanyl or carfentanil. As described above, the original Mann model (described with reference to FIG. 7) simulated antagonist administration 1-minute after the minute ventilation dropped to 40% baseline following administration of the synthetic opioid based on an approximate time needed to prepare and administer IM naloxone following detection of respiratory depression. To evaluate the impact of a potential delay in rescue on the adverse health effects, Laffont Model 700 was used to conduct simulations explored various times for antagonist administration (i.e., 0.5, 1, 2.5, 3, 5, 7.5 and 10 minutes) once the minute ventilation threshold of 40% was reached. In each scenario, the antagonist was given as a single IN dose of 3 mg nalmefene hydrochloride (corresponding to 2.7 mg nalmefene base) or 4 mg naloxone hydrochloride.

The current FDA approved intranasal doses of naloxone hydrochloride are chosen from 3, 4, and 8 mg in a 0.1 mL nasal spray. The current FDA approved intranasal doses of nalmefene is 2.7 mg in a 0.1 mL nasal spray. Additional variables evaluated in the study using simulations included the type of synthetic opioid (fentanyl, carfentanil), the dose of synthetic opioid, and the type of opioid use (chronic vs. naïve) by the victim.

Sub Study 1: Three Doses of Synthetic Opioids-Effects of Treatment with IN Nalmefene and IN Naloxone on Risk of Incidence of Cardiac Arrest

Three doses (similar to doses chosen under study II above) were tested for both synthetic opioids, including fentanyl doses of 1.63 mg and 2.97 mg, and corresponding carfentanil doses of 0.012 mg and 0.022 mg. The fentanyl doses of 1.63 mg and 2.97 mg were derived from post-mortem fentanyl concentrations measured in approximately 500 cases of fatal fentanyl overdose (National Drug Early Warning System, 2024; Mann et al., 2022) and the 2 mg dose considered potentially lethal by the Drug Enforcement Administration in the United States (Drug Enforcement Administration, 2024). Lower doses of 0.81 mg fentanyl and 0.010 mg carfentanil were also assessed as part of the sub study focused on including lower doses.

In the study, for each scenario, 2000 virtual subjects were simulated, accounting for between-subject variability and variability in pharmacodynamic binding parameters, as previously described in Study II. A bootstrap resampling method was used to estimate the summary statistics for prediction or risk of incidence of one of more potential adverse health effects including respiratory depression, cardiac arrest, brain hypoxia, etc. Here, the incidence of cardiac arrest was defined as total blood flow reaching 0.01 L/min. For example, summary statistics such as median and 2.5th and 97.5th percentiles delineating a 95% confidence interval were calculated as representative values of parameters associated with an adverse health effect.

Dosing scenarios were defined in the study based on the identity of the opioid that caused the overdose (e.g., fentanyl or carfentanil), amount of the opioid that caused the overdose, whether intervention was provided, identity of opioid antagonist delivered in the intervention if provided, the dose of opioid antagonist delivered in the intervention if provided, and the delivery time that the opioid antagonist was delivered in the intervention if provided (i.e., the delay in intervention being provided). A sample with size of 400 was drawn 2500 times from the 2000 virtual subjects simulated for each dosing scenario, and the incidence of cardiac arrest was estimated for each of the 2500 bootstrap samples. “Rescue rates” following IN naloxone and IN nalmefene administration were calculated using the following formula:

$$( \% {CA}_{no intervention} - \% {CA}_{intervention} ) / \% {CA}_{no intervention} \times 100$$

where CA represents the predicted incidence of cardiac arrest with or without intervention by way of delivering an opioid antagonist.

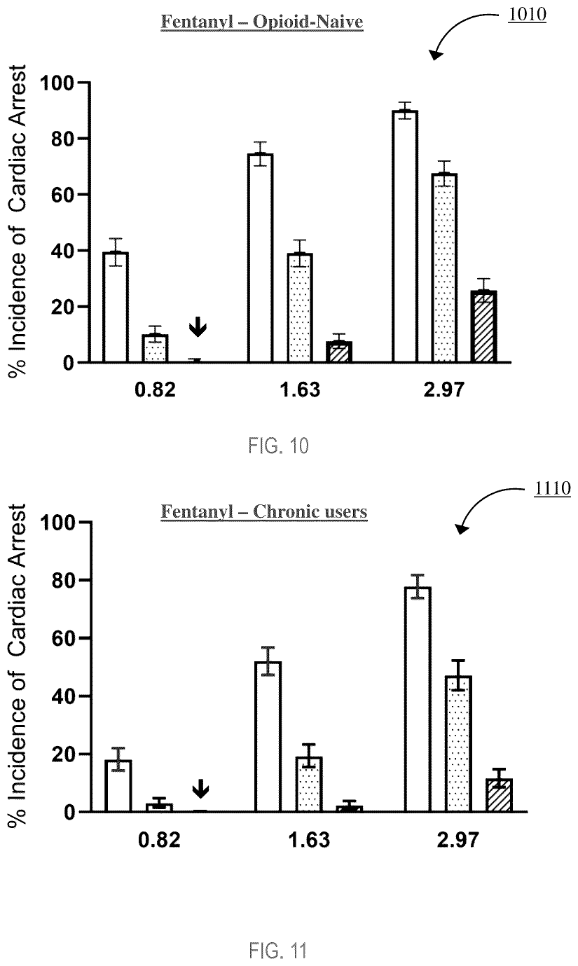

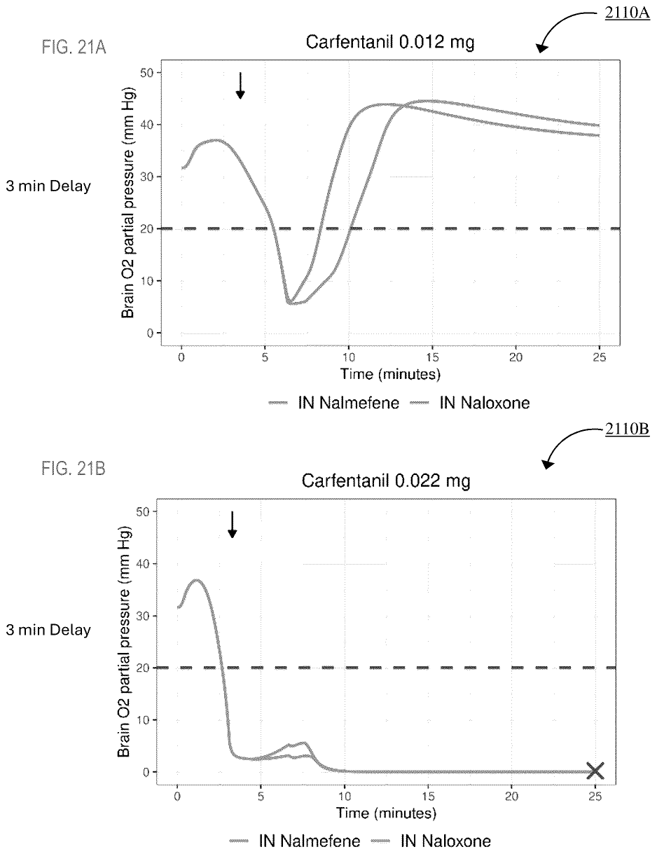

The example study compared the effectiveness of IN nalmefene and IN naloxone based on results from simulations conducted using the Laffont Model 700. FIGS. 10-21A and 21B illustrate some of the findings from the study.

FIG. 10 shows a bar graph 1010 illustrating predicted incidence of the adverse health effect of cardiac arrests in opioid-naïve subjects, following exposure to intravenous bolus doses of fentanyl, simulated using the Laffont model 700, without and with intervention using methods described herein, according to an embodiment. The bar graph 1010 shows the percentage of incidence without and after intervention using intranasal (IN) naloxone and IN nalmefene using methods disclosed herein, according to an embodiment.

FIG. 11 shows a bar graph 1110 illustrating predicted incidence of the adverse health effect of cardiac arrest in chronic opioid users, compared to the opioid-naïve counterparts in FIG. 10, also following exposure to intravenous bolus doses of fentanyl, simulated using the Laffont model 700, without and with intervention using methods described herein, according to an embodiment. The bar graph 1110 shows the percentage of incidence without and after intervention using opioid antagonists intranasal (IN) naloxone and IN nalmefene using methods disclosed herein, according to some embodiments.

The results shown in FIGS. 10 and 11 include percentage incidence of cardiac arrest in opioid-naïve subjects and chronic opioid users, respectively, when exposed to potentially lethal doses of 0.82 mg of fentanyl, indicated by the leftmost set of bars; 1.63 mg of fentanyl indicated by the middle set of bars; and 2.97 mg of fentanyl, indicated by the rightmost set of bars. Each bar from each set of bars corresponds to one of three conditions: (i) when no intervention was provided, indicated by clear, open bars; (ii) when intervention provided included reversal by delivering an intranasal (IN) pharmaceutical formulation including 4 mg naloxone hydrochloride, indicated by the bars with stippled pattern; and (iii) when the intervention provided included reversal by delivering an intranasal (IN) pharmaceutical formulation including 3 mg nalmefene hydrochloride (2.7 mg free base) indicated by the bars with the cross-hatched pattern. The values shown in FIGS. 10 and 11 represent the median percentage, and error bars indicate 95% confidence interval, of simulated subjects experiencing cardiac arrest after randomly sampling 400 out of 2000 virtual subjects 2500 times. Intervention by delivering pharmaceutical formulations with opioid antagonists, naloxone and nalmefene, were administered 1 minute after minute ventilation was reduced to 40% of baseline.

The IV doses of 1.63 and 2.97 mg fentanyl used in the study by Mann et al. (2022) were based on data from approximately 500 cases of fatal overdose and represent the mean and one standard deviation above the mean of plasma concentrations measured post-mortem, respectively. The incidence of cardiac arrest, which can be viewed as a surrogate measure of a fatality in the absence of intervention was >50% across all fentanyl simulations using the Mann model and the Laffont model 100 described herein. Based on estimates of the ratio of fatal:nonfatal opioid overdoses (1:5 to 1:14 across regions; see, Johnson, C. E., et al. (2024). Effects of States' Methods for Estimating Nonfatal Overdose, United States, 2021. Public Health Rep, 333549241263526. doi:10.1177/00333549241263526), the simulation studies were expanded to examine the effectiveness of IN naloxone and IN nalmefene using a lower dose of 0.82 mg of fentanyl in the example study.

As shown in FIGS. 10 and 11, a single dose of IN nalmefene in the form of 3 mg nalmefene hydrochloride, equivalent to 2.7 mg nalmefene, produced large and clinically meaningful reductions in the incidence cardiac arrest (see cross-hatched bars) compared to a single dose of IN naloxone in the form of 4 mg naloxone hydrochloride (stippled bars), following potentially lethal doses of fentanyl.

At the lower dose of 0.82 mg of fentanyl both a lower incidence of cardiac arrest and an increased effectiveness of both opioid antagonists, nalmefene and naloxone, were obtained as shown in FIGS. 10 and 11. The increased rescue rates reflected by the lowered incidence of cardiac arrests when exposed to the lower dose of 0.82 mg of fentanyl in the example study are highlighted by downward pointing arrow heads in FIGS. 10 and 11. In both opioid naïve subjects in the graph in FIG. 10 and in chronic opioid users shown in the graph in FIG. 11, the incidence of cardiac arrest produced by the 0.82 mg of fentanyl following rescue by nalmefene was <0.6% indicated by the downward pointing arrowheads (to show the position of the nalmefene bar in each set). As shown, the rescue rates were approximately 99% for IN nalmefene and 74-83% for IN naloxone (see, Table 13; FIGS. 10 and 11).

A similar trend was observed for carfentanil, a single dose of IN nalmefene in the form of 3 mg nalmefene hydrochloride, equivalent to 2.7 mg nalmefene, produced large and clinically meaningful reductions in the incidence cardiac arrest (see cross-hatched bars) compared to a single dose of IN naloxone in the form of 4 mg naloxone hydrochloride (stippled bars), following potentially lethal doses of carfentanil. The lowest dose of carfentanil (0.010 mg), however, was associated with lower rescue rates for both interventions using IN pharmaceutical formulation including nalmefene (88-97%) and IN pharmaceutical formulation including naloxone (44-66%) compared to the lowest dose of fentanyl (see Table 13).

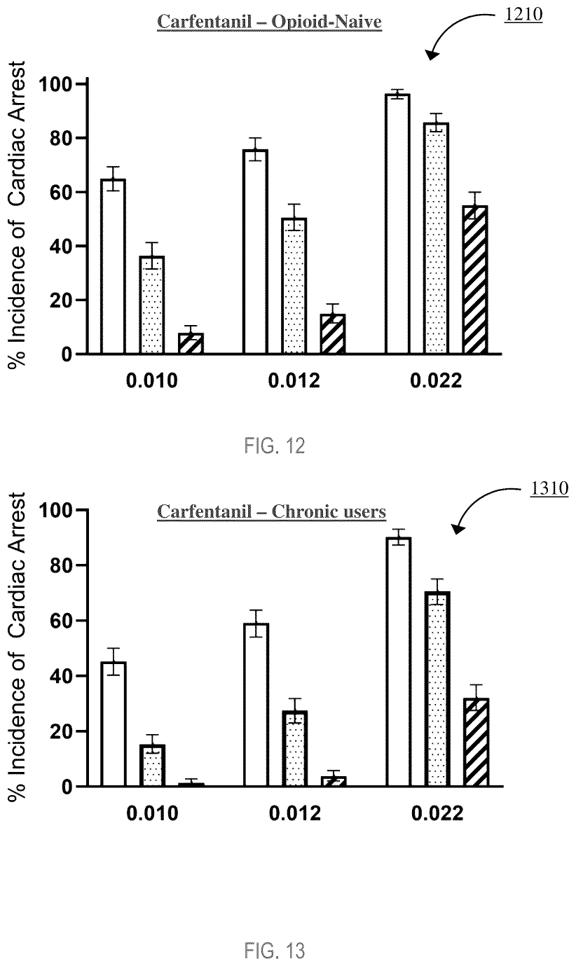

FIGS. 12 and 13 show percentage incidences of cardiac arrests in opioid-naïve (FIG. 12) and chronic opioid using (FIG. 13) subjects, exposed to three levels of potentially lethal doses of carfentanil: 0.010 mg of carfentanil, indicated by the leftmost set of bars; 0.012 mg of carfentanil indicated by the middle set of bars; and 0.022 mg of carfentanil, indicated by the rightmost set of bars. As in FIGS. 10 and 11, each bar from each set of bars corresponds to one of three conditions: (i) when no intervention was provided, indicated by clear, open bars; (ii) when intervention provided included reversal by delivering an intranasal (IN) pharmaceutical formulation including 4 mg naloxone hydrochloride, indicated by the bars with stippled pattern; and (iii) when the intervention provided included reversal by delivering an intranasal (IN) pharmaceutical formulation including 3 mg nalmefene hydrochloride (2.7 mg free base) indicated by the bars with the cross-hatched pattern. The values shown in FIGS. 12 and 13 represent the median percentage, and error bars indicate 95% confidence interval, of simulated subjects experiencing cardiac arrest after randomly sampling 400 out of 2000 virtual subjects 2500 times. Intervention by delivering pharmaceutical formulations with opioid antagonists, naloxone and nalmefene, were administered 1 minute after minute ventilation was reduced to 40% of baseline.

In the absence of intervention, carfentanil produced a dose-dependent increase in the incidence of cardiac arrest, with higher incidence rates compared to fentanyl as illustrated by comparing values in FIG. 12 to FIG. 10 for opioid naïve subjects and comparing values in FIG. 13 to FIG. 11 for chronic opioid using subjects. The use of IN nalmefene (cross-hatched bars) resulted in large and clinically meaningful reductions in the percentage of cardiac arrest compared to IN naloxone across all carfentanil dosing scenarios (FIG. 12). Rescue rates for 3 mg IN nalmefene and 4 mg IN naloxone are presented in Table 13.

TABLE 13
Predicted “Rescue Rates” with 3 mg IN Nalmefene
and 4 mg IN Naloxone Following a Synthetic Opioid Overdose
in Opioid-Naïve Subjects and Chronic Opioid Users

| | |
|---|---|
| Opioid-Naïve | Chronic Opioid Users |

| | | | |
|---|---|---|---|
| 3 mg | 4 mg | 3 mg | 4 mg |
| Nalmefene | Naloxone | Nalmefene | Naloxone |

Fentanyl

| | | | | |
|---|---|---|---|---|
| 0.82 mg | 98.5% | 74.4% | 99.4% | 83.4% |
| 1.63 mg | 89.8% | 47.7% | 95.8% | 63.1% |
| 2.97 mg | 71.5% | 25.0% | 85.1% | 39.5% |

Carfentanil

| | | | | |
|---|---|---|---|---|
| 0.01 mg | 87.8% | 44.0% | 96.9% | 65.9% |
| 0.012 mg | 80.4% | 33.5% | 93.6% | 53.5% |
| 0.022 mg | 42.9% | 11.0% | 64.5% | 21.7% |

As shown in the graphs in FIGS. 10-13, the results indicated that while both opioid antagonists were effective at reducing risk of incidence of the adverse health effect of cardiac arrest at the lower dose of 0.82 mg fentanyl, clear differences were demonstrable in favor of nalmefene, either when carfentanil was administered or when the dose of fentanyl and carfentanil was increased. Rescue rates from carfentanil induced opioid overdoses were typically lower in opioid-naïve subjects compared to chronic opioid users across all three sets.

The higher rescue rates observed in simulations using lower doses of synthetic opioids may, in part, reconcile reports of very high rescue rates (>99%) by first responders with data from the Centers for Disease Control (CDC) indicating that on average, 23.7% of decedents (up to 40.8% in some jurisdictions) with a presumed overdose had received some form of naloxone (Centers for Disease Control and Prevention, 2024). In total, these findings demonstrate that the greatest differences in effectiveness between nalmefene and naloxone are manifested in simulations using high doses of fentanyl and carfentanil, scenarios which put an overdose victim at highest risk of a fatal overdose.

Sub Study II: Time Critical Nature of Delivering Intervention with IN Nalmefene and IN Naloxone on Risk of Incidence of Cardiac Arrest

The sub study assesses the impact of a delay in intervention on the risk of incidence of cardiac arrest suffered by virtual subjects who were opioid naïve and chronic opioid users. following an overdose with fentanyl or carfentanil. The Laffont Model 700 was used to conduct simulations exploring various times for intervention with antagonist administration including at time points of 0.5, 1, 2.5, 3, 5, 7.5 and 10 minutes following the first time point when minute ventilation threshold of 40% was reached. In each scenario, the antagonist was given as a single dose IN pharmaceutical formulation including 3 mg nalmefene hydrochloride (corresponding to 2.7 mg nalmefene base) or 4 mg naloxone hydrochloride.

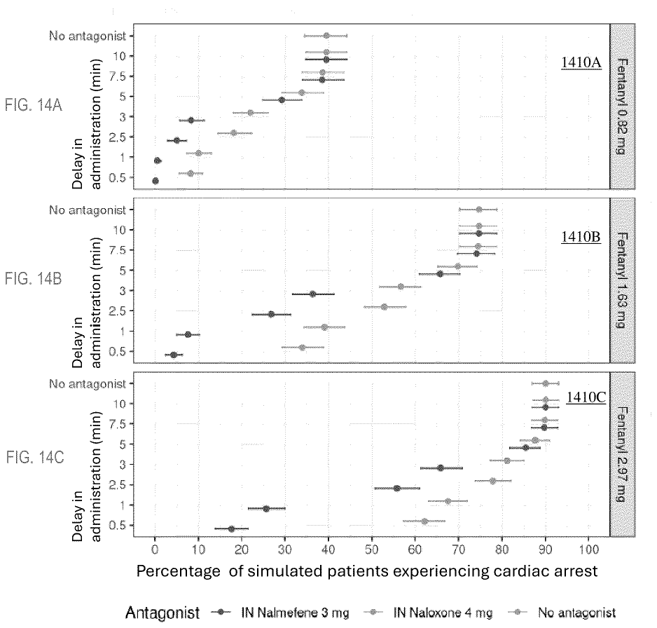

FIGS. 14A-14C, 15A-15C, 16A-16C, and 17A-17C illustrate the time critical nature of intervention following a synthetic opioid overdose using the three different amounts of fentanyl and carfentanil described above. The plots show the impact of varying the delay in administration of IN opioid antagonists following potentially lethal doses of fentanyl and carfentanil in both opioid-naïve individuals and chronic opioid users. Each plot shows median percentage, and error bars indicating 95% confidence interval, of simulated subjects experiencing cardiac arrest after randomly sampling 400 out of 2000 virtual subjects 2500 times. Simulations were conducted using the parameters described in in reference to FIGS. 10-13 except that intervention in the form of delivering IN pharmaceutical formulations including opioid antagonists 4 mg naloxone hydrochloride, or nalmefene hydrochloride 3 mg were administered at varying delivery time between 0.5-10 minutes (ordinate) after minute ventilation reduced below an identified threshold value of 40% of baseline following IV bolus doses of either fentanyl or carfentanil. The abscissa in each plot indicates percentage incidence of the adverse health effect of a cardiac arrest as a function of the ordinate indicating delay in intervention provided in minutes. In each plot, blue markers indicate data from subjects administered with IN pharmaceutical formulations including 3 mg nalmefene hydrochloride, orange markers indicate data from subjects administered with IN pharmaceutical formulations including 4 mg naloxone hydrochloride, and gray markers indicate data from subjects who did not receive any intervention in the form of a pharmaceutical formulation including an opioid antagonist.

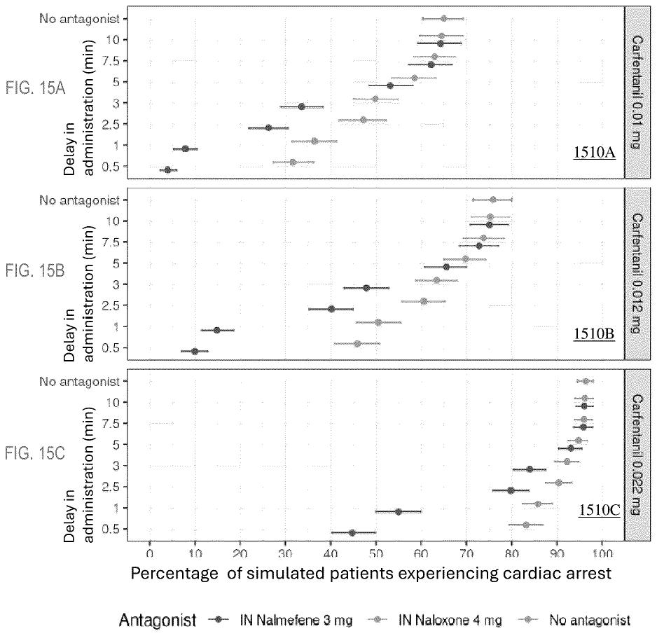

FIGS. 14A-14C illustrates the time critical nature of intervention following a fentanyl overdose using three different amounts of fentanyl—0.82 mg in plot 710A in FIG. 14A, 1.63 mg in plot 1410B in FIG. 14B, and 2.97 mg fentanyl in plot 1410C in FIG. 14C, using simulations in opioid-naïve subjects. FIGS. 15A-15C illustrate the time critical nature of intervention following a carfentanil overdose using three different amounts of carfentanil—0.010 mg in plot 1510A in FIG. 15A, 0.012 mg carfentanil in plot 1510B in FIG. 15B, and 0.022 mg carfentanil in plot 1510C in FIG. 15C, using simulations in opioid-naïve subjects.

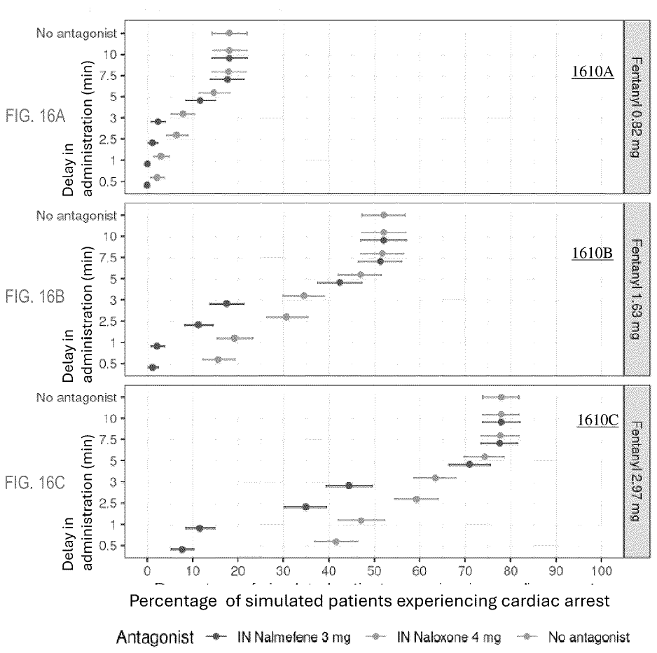

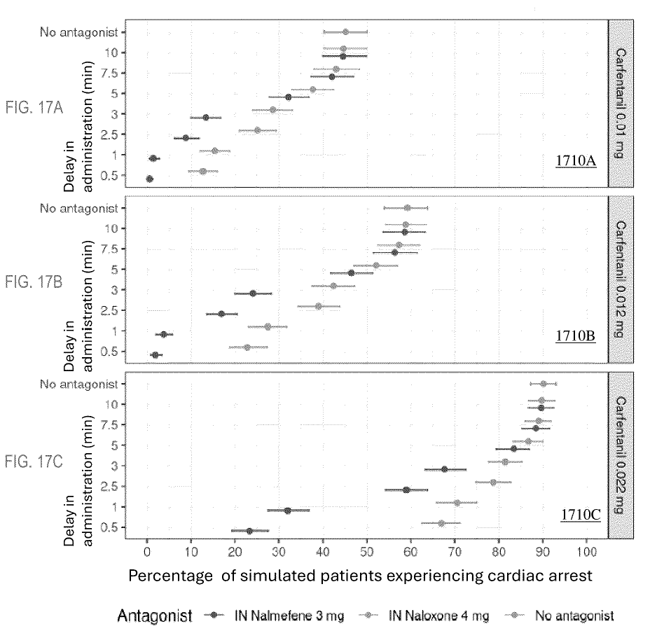

FIGS. 16A-16C illustrates the time critical nature of intervention following a fentanyl overdose using the three different amounts of fentanyl in chronic opioid using subjects—0.82 mg in plot 1610A in FIG. 16A, 1.63 mg in plot 1610B in FIG. 16B, and 2.97 mg fentanyl in plot 1610C in FIG. 16C. Correspondingly, FIGS. 17A-17C illustrate the time critical nature of intervention following a carfentanil overdose using the three different amounts of carfentanil in chronic opioid using subjects—0.010 mg in plot 1710A in FIG. 17A, 0.012 mg carfentanil in plot 1710B in FIG. 17B, and 0.022 mg carfentanil in plot 1710A in FIG. 17C, using simulations in opioid-naïve subjects.

Across the simulations, a more rapid intervention, for example, intervention by way of delivering an opioid antagonist within 0.5 minutes after the time of reduction in the minute ventilation below a threshold of 40% of baseline, resulted in numerically modest reductions in the risk of incidence of cardiac arrest for subjects receiving either of the two opioid antagonists—3 mg nalmefene and 4 mg naloxone. The reduction in risk of incidence of cardiac arrest increased or reached statistical significance (indicated by no overlap in the 95% CIs), compared to a 1-minute delay in intervention following nalmefene administration in the more severe dosing scenarios, that is, following the highest dose of 2.97 mg fentanyl and 0.022 mg of carfentanil (compare blue markers in plots in FIGS. 14C and 16C for highest dose of fentanyl and compare blue markers in plots in FIGS. 15C and 17C for highest dose of carfentanil).

Increasing the delay in administration of the antagonist reduced the effectiveness of both IN naloxone and IN nalmefene, manifested as a time-dependent increase in the incidence of cardiac arrest across simulations. Following simulated fentanyl and carfentanil overdoses, the incidence of cardiac arrest was lower (no overlap in the 95% CI) following IN nalmefene compared to IN naloxone until the intervention was delayed for 2.5-3 minutes. A delay of 5 minutes resulted in large reductions in the effectiveness of both nalmefene and naloxone across dosing scenarios. Introducing delays ≥7.5 minutes resulted in a loss in effectiveness of both agents (see FIGS. 14A-14C, 15A-15C, 16A-16C, and 17A-17C).

Sub Study III: Effects of Delivering Intervention with IN Nalmefene and IN Naloxone on Risk of Incidence of Brain Hypoxia from Synthetic Opioid Overdose

The sub study assesses the impact of a delay in intervention on the duration of brain hypoxia suffered by a typical or mean virtual subject with chronic opioid use. Brain tissue oxygen partial pressure (PbtO2) was one of the variables simulated by the model, and the duration of hypoxia was defined as the time during which PbtO2 was below the critical threshold of 20 mm Hg following an overdose with fentanyl or carfentanil.

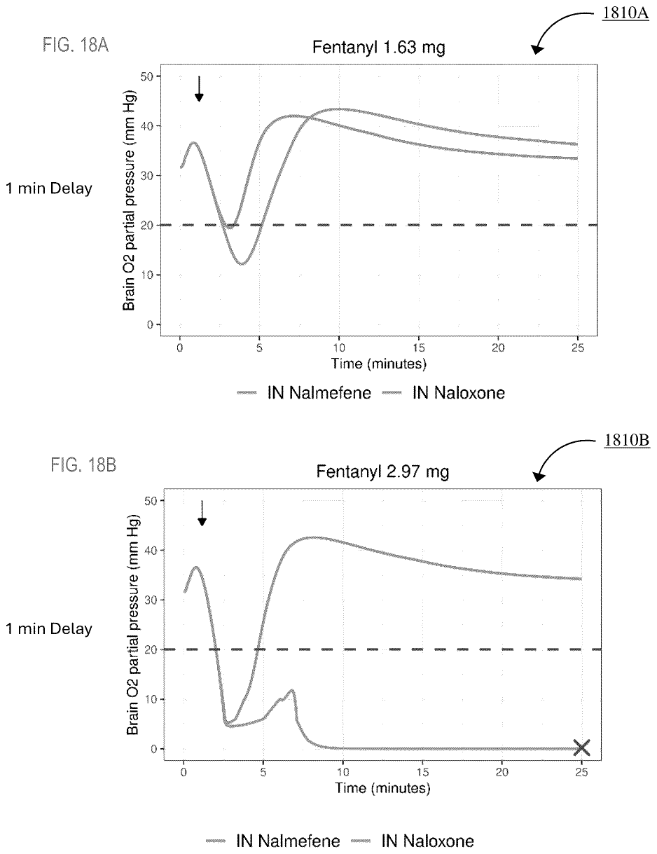

FIGS. 18A-18B, 19A-19B, 20A-20B, and 21A-21B illustrate results from assessing the effect of 3 mg IN nalmefene and 4 mg IN naloxone in limiting the duration of brain hypoxia following a synthetic opioid overdose, as described herein. The Laffont model 700 was used to conduct simulations to assess effect of delivering IN pharmaceutical formulations including naloxone and nalmefene in reducing the duration of brain hypoxia resulting from the overdose.

The simulations were conducted in a manner like those described previously with reference to the simulations to assess effect of intervention in the form of delivering IN pharmaceutical formulations including naloxone and nalmefene on risk of incidence of cardiac arrest. The simulations were conducted in a “typical” (mean) subject with chronic opioid use. Brain hypoxia was defined as the time brain tissue oxygen partial pressure (PbtO₂) falls below an identified critical threshold of 20 mm Hg (see, Lund, A., et al. Brain hypoxia and metabolic crisis are common in patients with acute brain injury despite a normal intracranial pressure. Scientific Reports, 14(1), p. 23828). The simulations were conducted to compare effects of IN nalmefene and IN naloxone in reversing effects of brain hypoxia from exposure to two different amounts of fentanyl and carfentanil with intervention provided at two different time points (1 min and 3 mins).

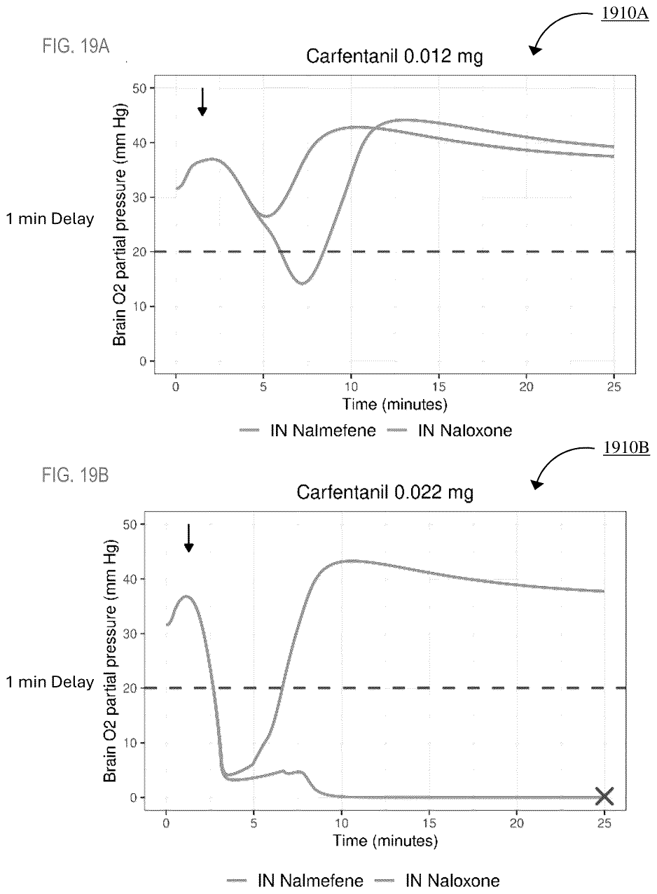

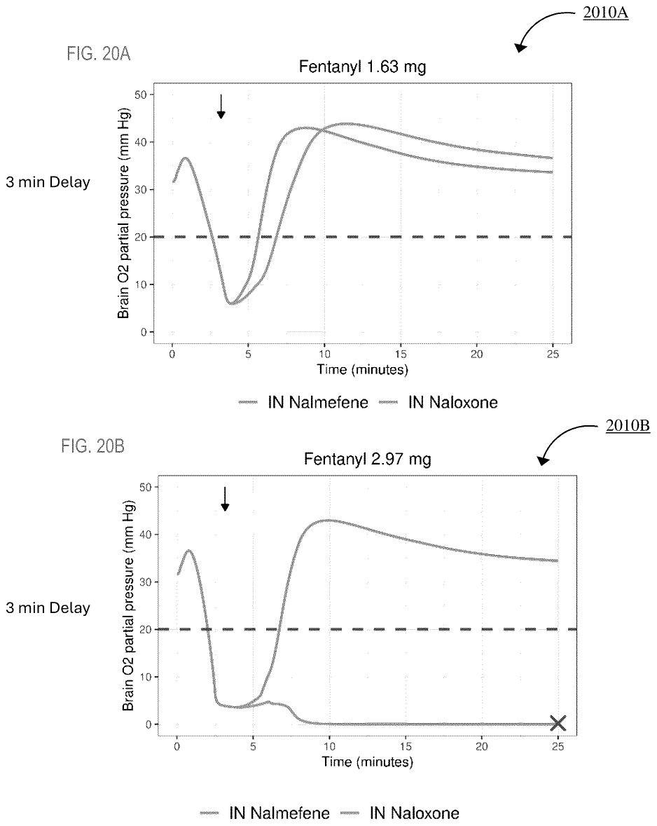

The results are shown in plots 1810A and 1810B in FIGS. 18A and 18B for data from 1.63 mg and 2.97 mg fentanyl overdoses, respectively, treated with intervention at 1 min, and in plots 2010A and 2010B in FIGS. 20A-20B for data from 1.63 mg and 2.97 mg fentanyl overdoses, respectively, treated with intervention at 3 min. Similarly, plots 1910A and 1910B in FIGS. 19A and 19B show data from 0.012 mg and 0.022 mg carfentanil overdoses, respectively, treated with intervention at 1 min, and plots 2110A and 2110B in 21A-21B show data from 0.012 mg and 0.022 mg carfentanil overdoses, respectively, treated with intervention at 3 min. Plots show evolution of O₂ partial pressure (PbtO₂) in brain tissue as a function of time, following fentanyl or carfentanil administration at 0 minutes and following intervention delivered at a time indicated by the downward pointing arrow. Blue traces indicate effects from intervention in the form of administering an IN pharmaceutical formulation including nalmefene and red traces indicate effects from intervention in the form of administering an IN pharmaceutical formulation including naloxone. The plots also show the critical threshold of 20 mm Hg partial pressure at, or below which brain hypoxia occurs indicated by the dashed horizontal line. The red X designates when a typical virtual subject had a cardiac arrest.

As shown, with a 1-minute delay in antagonist administration, following exposure to the medium dose of 1.63 mg fentanyl, brain hypoxia lasted for approximately 0.51 minutes when treated with IN nalmefene, indicated by the portion of blue trace at or below the critical threshold indicated by the dashed line in FIG. 18A. In comparison, brain hypoxia lasted for 2.46 minutes when treated with IN naloxone, as indicated by the portion of red trace at or below the critical threshold indicated by the dashed line in plot 1810A in FIG. 18A. As shown in FIG. 19A, on the other hand, at the medium dose of 0.012 mg carfentanil, brain PbtO₂ did not fall below 20 mm Hg when IN nalmefene was administered with a 1-minute delay, compared to 2.45 minutes of brain hypoxia following IN naloxone administration (compare blue and red traces in plot 1910A in FIG. 19A with reference to the critical threshold indicated by the horizontal red dashed line).

When administered with a 1-minute delay, IN nalmefene was also more effective at reducing the duration of brain hypoxia at the high dose of 2.97 mg fentanyl (see plot 1810B in FIG. 18B) and the high dose of 0.022 mg of carfentanil (see plot 1910B in FIG. 19B). At these high doses, administration of IN naloxone in a typical virtual subject resulted in cardiac arrest, which was only observed for IN nalmefene following administration of 0.022 mg carfentanil with a 3-minute delay.

### Conclusion

The sub studies explore the effectiveness of IN naloxone compared to IN nalmefene in three domains: (i) following an overdose with a variable quantity of synthetic opioid; (ii) varying the time between overdose and rescue; and (iii) reducing the duration of brain hypoxia following a synthetic opioid overdose.

Findings from the example sub study I showed that a single dose of an IN pharmaceutical formulation including 3 mg nalmefene hydrochloride, equivalent to 2.7 mg nalmefene, produced significant and clinically meaningful reductions in the incidence cardiac arrest compared to a single dose of IN pharmaceutical formulation including 4 mg naloxone hydrochloride, following potentially lethal doses of variable amounts of synthetic opioids fentanyl and carfentanil (see FIGS. 10-13).

Findings from sub study 11 showed that the introduction of even modest delays between the detection of opioid overdose, through visible symptoms like respiratory distress, and the rescue efforts provided through intervention resulted in a rapid decline in the effectiveness of both IN naloxone and IN nalmefene (FIGS. 14A-14C, 15A-15C, 16A-16C, and 17A-17C). These findings underscore both the rapid onset of respiratory depression produced by synthetic opioids and the need for prompt intervention following a potentially lethal overdose. The challenges in recognizing the signs of an opioid overdose in a community setting, together with data indicating that potential bystanders were present in less than half of all overdoses (per data from the Centers for Disease Control and Prevention), further emphasizes the importance of examining the effect of delays in administering IN nalmefene compared to IN naloxone. The results show that an IN pharmaceutical formulation including 3 mg nalmefene resulted in a lower incidence of cardiac arrest compared to an IN pharmaceutical formulation including 4 mg IN naloxone when the delay in intervention ranged between 0.5-3 minutes. The ability of both opioid antagonists to reduce the incidence of cardiac arrest, however, was reduced for delays ≥7.5 minutes.

Findings from sub study III showed that the duration of brain hypoxia, defined as brain oxygen levels below a critical threshold of 20 mm Hg, experienced by simulated overdose subjects was sensitive to the type and time of intervention provided by either IN pharmaceutical formulation including naloxone or IN pharmaceutical formulation including nalmefene. When IN pharmaceutical formulations with opioid antagonists were administered 1-minute after minute ventilation was reduced to 40% of baseline in a typical chronic opioid user, IN nalmefene was more effective than IN naloxone in reducing the duration of hypoxia across simulation scenarios. (FIGS. 18A-18B and FIGS. 19A-19B). This was especially apparent at the higher doses of synthetic opioids when naloxone was unable to produce a meaningful reduction in the duration of hypoxia (FIGS. 18B and 19B). Delaying intervention by >5 minutes abolished the ability of both opioid antagonists to reduce the duration of hypoxia, consistent with the rapid onset of respiratory depression produced by a potentially fatal synthetic opioid overdose.

Periods of hypoxia associated with non-fatal overdoses have the potential to cause enduring functional impairment and brain damage, making rapid restoration of breathing critical for minimizing the potential for long-term neurologic consequences. For individuals with opioid use disorder who are likely to experience recurrent episodes of prolonged opioid-induced respiratory depression, the damage is cumulative and may lead to the development of important disability, including mental and motor dysfunction. Hence, while the primary objective of overdose reversal is to prevent death, the potential for neurological damage is to be considered, both in opioid-naïve and chronic opioid users, when evaluating an optimal overdose reversal strategy. This need emphasizes the superior capacity of IN pharmaceutical formulations including nalmefene to induce recovery compared the effects of IN pharmaceutical formulations including other opioid antagonists like naloxone.

The example studies provided herein are representative examples and not meant to be limiting. Cardiac arrest was used as an example endpoint in the simulations because in the absence of intervention, the cardiovascular consequences of sustained respiratory depression are inevitably fatal.

The representative studies disclosed herein show with simulations results from IV doses of fentanyl and carfentanil, which correspond to worst-case or extremely severe, near fatal or fatal scenarios. Synthetics are also smoked and ingested, most often unintentionally in the form of counterfeit pills. The methods disclosed herein can be suitably used to evaluate effects of opioid overdose following inhalation and ingestion that could potentially expand the window of opportunity for successful intervention compared to an IV bolus because of differences in the speed of absorption and bioavailability of synthetics such as fentanyl following inhalation and ingestion.

Although the present invention has been described with reference to specific details of certain embodiments thereof in the above examples, it will be understood that modifications and variations are encompassed within the spirit and scope of the invention.

1. A method of treating opioid overdose in a human by administering to the human in need thereof a single dose of an intranasal pharmaceutical formulation comprising at least one pharmaceutically acceptable carrier and consisting of a therapeutically effective amount of nalmefene opioid antagonist, to reduce a predicted risk of an adverse health effect resulting from the opioid overdose for the human following the administration of said intranasal pharmaceutical formulation compared to a predicted risk of said adverse health effect for the human following the administration of a single dose of an intranasal pharmaceutical formulation comprising a pharmaceutically acceptable carrier and consisting of a therapeutically effective amount of naloxone opioid antagonist.

2. An intranasal pharmaceutical formulation for treatment of opioid overdose in a human in need thereof, comprising at least one pharmaceutically acceptable carrier and consisting of a therapeutically effective amount of nalmefene opioid antagonist, to further reduce a predicted risk of an adverse health effect for the human compared to a predicted risk of incidence of an adverse health effect for the human from an intranasal pharmaceutical formulation for treatment of opioid overdose in a human in need thereof comprising at least one pharmaceutically acceptable carrier and consisting of a therapeutically effective amount of naloxone opioid antagonist.

3. The method of claim 1, wherein the nalmefene is nalmefene hydrochloride.

4. The method of claim 1, wherein the nalmefene is nalmefene free base.

5. The method of claim 1, wherein the adverse health effect is chosen from at least one of respiratory depression, cardiac arrest, brain hypoxia, and death.
